(19) United States
(12) Patent Application Publication
(10) Pub. No.: US 2007/0025965 A1
(43) Pub. Date: Feb. 1, 2007

(54) Therapies for renal failure using interferon-beta

(76) Inventor: Roy R. Lobb, Westwood, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 10/521,513

(22) PCT Filed: Jul. 17, 2003
(86) PCT No.: PCT/US03/22440

§ 371 Date: Nov. 18, 2005

Related U.S. Application Data
(60) Provisional application No. 60/396,393, filed on Jul. 17, 2002.

(51) Int. Cl. A61K 38/21

(52) U.S. Cl. 424/85.5

(57) ABSTRACT

The present invention provides methods for the treatment, and pharmaceuticals for the use in the treatment, of mammalian subjects having, or at risk of developing, glomerulonephritis or chronic renal failure. The methods involve the administration of IFN-β therapeutics.

### BACKGROUND OF THE INVENTION

Chronic renal failure (CRF) may be defined as a progressive, permanent and significant reduction of glomerular filtration rate (GFR) due to a significant and continuing loss of nephrons. Chronic renal failure typically begins from a point at which a chronic renal insufficiency (i.e., a permanent decrease in renal function of at least 50-60%) has resulted from some insult to the renal tissues that has caused a significant loss of nephron units. The initial insult may or may not have been associated with an episode of acute renal failure or it may be associated with any number of renal disorders including, but not limited to, end-stage renal disease, chronic diabetic nephropathy, diabetic glomerulopathy, diabetic renal hypertrophy, hypertensive nephrosclerosis, hypertensive glomerulosclerosis, chronic glomerulonephritis, hereditary nephritis, renal dysplasia and chronic rejection following renal allograft transplantation. Irrespective of the nature of the initial insult, chronic renal failure manifests a “final common path” of signs and symptoms as nephrons are progressively lost and GFR progressively declines. This progressive deterioration in renal function is slow, typically spanning many years or decades in human patients, but seemingly inevitable.

In humans, as chronic renal failure progresses, and GFR continues to decline to less than 10% of normal (e.g., 5-10 ml/min), the subject enters end-stage renal disease (ESRD). During this phase, the inability of the remaining nephrons to adequately remove waste products from the blood, while retaining useful products and maintaining fluid and electrolyte balance, leads to a decline in which many organ systems, and particularly the cardiovascular system, may rapidly begin to fail. At this point, renal failure will rapidly progress to death unless the subject receives renal replacement therapy (i.e., chronic hemodialysis, continuous peritoneal dialysis, or kidney transplantation).

One renal disease that can lead to CRF is glomerulonephritis. Glomerulonephritis is characterized by inflammation and resulting enlargement of the glomeruli that is typically due to immune complex formation. The accumulation of immune complexes in the glomeruli results in inflammatory responses, involving inter alia hypercellularity, that can cause total or partial blockage of the glomerulus through, among other factors, narrowing of capillary lumens. One result of this process is the inhibition of the normal filtration function of the glomerulus. Blockage may occur in large numbers of glomeruli, directly compromising kidney function and often causing the abnormal deposition of proteins in the walls of the capillaries making up the glomerulus. Such deposition can, in turn, cause damage to glomerular basement membranes. Those glomeruli that are not blocked develop increased permeability, allowing large amounts of protein to pass into the urine, a condition referred to as proteinuria.

In many cases of severe glomerulonephritis, pathological structures called crescents are formed within the Bowman's space, further impeding glomerular filtration. These structures can only be seen by microscopic examination of tissue samples obtained by biopsy or necropsy, and are thus not always observed in those patients in which they occur. Crescents are a manifestation of hypercellularity and are thought to arise from the extensive abnormal proliferation of parietal epithelial cells, the cells that form the inner lining of the Bowman's capsule. Clinical research has shown that there is a rough correlation between the percentage of glomeruli with crescents and the clinical severity of the disease, and thus the patient's prognosis. When present in large numbers, crescents are a poor prognostic sign.

Approximately 600 patients per million receive chronic dialysis each year in the United States, at an average cost approaching $60,000-$80,000 per patient per year. Of the new cases of end-stage renal disease each year, approximately 28-33% are due to diabetic nephropathy (or diabetic glomerulopathy or diabetic renal hypertrophy), 24-29% are due to hypertensive nephrosclerosis (or hypertensive glomerulosclerosis), and 15-22% are due to glomerulonephritis. The 5-year survival rate for all chronic dialysis patients is approximately 40%, but for patients over 65, the rate drops-to approximately 20%. A need exists, therefore, for treatments which will prevent the progressive loss of renal function which has caused almost two hundred thousand patients in the United States alone to become dependent upon chronic dialysis, and which results in the premature deaths of tens of thousands each year.

### SUMMARY OF THE INVENTION

In one embodiment, the invention provides a method for treating glomerulonephritis or chronic renal failure in a mammal having or likely to develop glomerulonephritis, comprising administering to the mammal a therapeutically effective amount of an IFN-β therapeutic. The invention also provides uses of IFN-β therapeutics in the manufacture of a medicament for the treatment or prevention of glomerulonephritis. The glomerulonephritis can be selected from the group consisting of focal glomeruloscerosis, collapsing glomerulopathies, minimal change disease, crescentic glomerulonephritis, nephritic syndrome, nephrotic syndrome, primary glomerulonephritis, secondary glomerulonephritis, proliferative glomerulonephritis, membraneous glomerulonephritis, membranoproliferative glomerulonephritis, immune-complex glomerulonephritis, anti-glomerular basement membrane (anti-GBM) glomerulonephritis, pauci-immune glomerulonephritis, diabetic glomerulopathy, chronic glomerulonephritis, and hereditary nephritis. The IFN-β may be mature or immature and may lack the initiator methionine. The IFN-β may be human IFN-β, e.g., IFN-β-1a and IFN-β-1b. The IFN-β may be a protein that is at least about 95% identical to full length mature human IFN-β having SEQ ID NO: 4. The IFN-β may be full length mature human IFN-β comprising or consisting of SEQ ID NO: 4. The IFN-β may be glycosylated or non-glycosylated. The IFN-β therapeutic may also be full length mature human IFN-β comprising SEQ ID NO: 4 fused to the constant domain of a human immunoglobulin molecule, e.g., the heavy chain of IgGl. For example, IFN-β therapeutic may comprise SEQ ID NO: 14. The IFN-β therapeutic may also comprise a pegylated IFN-β.

The IFN-β therapeutic may comprise a stabilizing agent, which may be an acidic amino acid. It may also be arginine. The IFN-β therapeutic may have a pH between about 4.0 and 7.2. In a preferred embodiment, the IFN-β therapeutic is AVONEX®.

The IFN-β therapeutic may be administered parenterally, e.g., intravenously (i.v.), subcutaneoulsy and intramuscularly (i.m.). The method may comprise administering to the mammal several doses of an IFN-β therapeutic. The IFN-β therapeutic may be administered over several days. For example, it may be administered weekly at a dose of 6 MIU. It may also be administered three times a week at a dose of 3, 6 or 12 MIU. Administration of an IFN-β therapeutic may reduce, e.g., proteinuria, glomerular cell proliferation or glomerular inflammation in the mammal.

In a preferred embodiment, the mammal is a human. The human may be a patient. The mammal may be a mammal that is likely to develop glomerulonephritis as indicated, e.g., by signs of an upcoming inflammation of at least one glomerulus. The mammal may be a mammal that is likely to develop chronic renal failure or has chronic renal failure as indicated, e.g., by the presence of chronic renal insufficiency. A mammal identified as having glomerulonephritis by the presence of an inflammation of at least one glomerulus; glomerular hypertrophy; tubular hypertrophy; glomerulosclerosis; or tubulointerstitial sclerosis. In certain embodiments, the mammal is not a mammal that harbors a virus, e.g., a hepatitis virus, such as hepatitis B or C, causing glomerulonephritis or wherein the glomerulonephritis was caused by a virus. In other embodiments, the mammal does not have end-stage renal failure or renal cell carcinoma.

### BRIEF DESCRIPTION OF THE FIGURES

FIG. 1 shows the nucleotide (SEQ ID NO: 11) and amino acid (SEQ ID NO: 12) sequences of a fusion protein consisting of the VCAM signal sequence fused to the mature full length human IFN-β (SEQ ID NO: 3 and 4), in which the glycine at amino acid 162 of SEQ ID NO: 4 is replaced with a cysteine, fused to the hinge, CH2 and CH3 domains of human IgGlFc (ZL5107).

FIG. 2 shows the nucleotide (SEQ ID NO: 13) and amino acid (SEQ ID NO: 14) sequences of a fusion protein consisting of the VCAM signal sequence fused to the mature full length human IFN-β (SEQ ID NO: 3 and 4), in which the glycine at amino acid 162 of SEQ ID NO: 4 is replaced with a cysteine; fused to the G4S linker which is fused to the hinge, CH2 and CH3 domains of human IgGlFc (ZL6206).

FIG. 3 shows the level of proteinuria at days 7, 14, 21 and 28 in rats having nephrotoxic nephritis (NTN) treated with 3×10⁵ units rat IFN-β per day, 6×10⁵ units rat IFN-β per day or vector alone (“control”) for 6 days per week starting at day 0.

FIG. 4 shows the level of proteinuria at days 7, 14, 21 and 28 in rats having nephrotoxic nephritis (NTN) treated with 6×10⁵ units rat IFN-β per day or vector alone (“control”) for 6 days per week starting at day 0.

FIG. 5 shows the number of proliferationg cells from glomeruli in rats having nephrotoxic nephritis (NTN) treated with 6×10⁵ units rat IFN-β per day or vector alone (“RSA”) for 6 days per week from day 0 to day 7.

FIG. 6 shows the level of proteinuria at days 7 and 10 in rats having Thy 1 glomerulonephritis treated with 6×10⁵ units rat IFN-β per day or vector alone (“RSA”) for 6 days per week stag at day 0 to day 10.

FIG. 7 shows the level of creatine clearance at days 7 or 10 in rats having Thy 1 glomerulonephritis treated with 6×10⁵ units rat IFN-β per day or vector alone (“RSA”) for 6 days per week starting at day 0 to day 10.

FIG. 8 shows the glomerular proliferation score at day 10 in rats having Thy 1 glomerulonephritis treated with 6×10⁵ units rat IFN-β per day or vector alone (“RSA”) for 6 days per week starting at day 0 to day 10.

FIG. 9 shows the level of proteinuria at days 7 and 14 in rats having puromycin aminonucleoside nephropathy (PAN) treated with 6×10², 6×10³, 6×10⁴, or 6×10⁵ units rat IFN-β per day or vector alone (“control”).

### DETAILED DESCRIPTION OF THE INVENTION

The invention is based at least in part on the discovery that at least certain symptoms of glomerulonephritis in a mammal can be improved by administration of INF-β to the mammal. In particular, it has been observed that proteinuria, glomerular cell proliferation and inflammation are significantly reduced by administration of IFN-β. Accordingly, the invention provides methods and compositions for treating glomerulonephritis in mammals.

1. Definitions:

To more clearly and concisely point out the subject matter of the claimed invention, the following definitions are provided for specific terms used in the written description and the appended claims.

As used in the specification and the appended claims, the singular forms “a,” “an” and “the” include plural referents unless the context clearly dictates otherwise.

The “glomerular filtration rate” or “GFR” is proportional to the rate of clearance into urine of a plasma-borne substance which is not bound by serum proteins, is freely filtered across glomeruli, and is neither secreted nor reabsorbed by the renal tubules. Thus, as used herein, GFR preferably is defined by the following equation:

$$GFR = \frac{{U}_{conc} \times V}{{P}_{conc}}$$

where U_{conc} is the urine concentration of the marker, P_{conc} is the plasma concentration of the marker, and V is the urine flow rate in ml/min. Optionally, GFR is corrected for body surface area. Thus, the GFR values used herein may be regarded as being in units of ml/min/1.73m². The preferred measure of GFR is the clearance of insulin but, because of the difficulty of measuring the concentrations of this substance, the clearance of creatinine is typically used in clinical settings. For example, for an average size, healthy human male (70 kg, 20-40 yrs), a typical GFR measured by creatinine clearance is expected to be approximately 125 ml/min with plasma concentrations of creatinine of 0.7-1.5 mg/dL. For a comparable, average size woman, a typical GFR measured by creatinine clearance is expected to be approximately 115 ml/min with creatinine levels of 0.5-1.3 mg/dL. During times of good health, human GFR values are relatively stable until about age 40, when GFR typically begins to decrease with age. For subjects surviving to age 85 or 90, GFR may be reduced to 50% of the comparable values at age 40. An estimate of the “expected GFR” or “GFRₑₓₚ” may be provided based upon considerations of a subject's age, weight, sex, body surface area, and degree of musculature, and the plasma concentration of some marker compound (e.g., creatinine) as determined by a blood test. Thus, as an example, an expected GFR or GFRₑₓₚ may be estimated as:

$${GFR}_{exp} \approx \frac{( 140 - age ) \times weight ( kg )}{72 \times {P}_{conc} ( mg \text{/} dl )}$$

This estimate does not take into consideration such factors as surface area, degree of musculature, or percentage body fat. Nonetheless, using plasma creatinine levels as the marker, this formula has been employed for human males as an inexpensive means of estimating GFR. Because creatinine is produced by striated muscle, the expected GFR or GFPₑₓₚ of human female subjects is estimated by the same equation multiplied by 0.85 to account for expected differences in muscle mass. (See Lemann, et al. (1990) Am. J. Kidney Dis. 16(3):236-243.)

“Glomerulonephritis,” “nephritis,” “acute nephritis” and “glomerular nephritis” are used interchangeably herein.

“IFN-β-1a” refers to an IFN-β molecule having the amino acid sequence of the wild-type human IFN-β and is glycosylated.

“IFN-β-1b” refers to an IFN-β molecule having the amino acid sequence of the wild-type IFN-β, wherein the cysteine at position 17 is replaced with a serine; the methione at position 1 (“initiator methionine”) is lacking and the molecule is not glycosylated.

“IFN-β variant” refers to a wild-type IFN-β protein having one or more modifications, e.g., amino acid deletions, additions, substitutions, a posttranslational modification or including one or more non-naturally occurring amino acid residues or linkages between them. Portions of IFN-βs are included in the term “IFN-β variant.” A “biologically active IFN-β variant” is an IFN-β variant that has at least some activity in treating renal disorders, e.g. glomerulonephritis. An IFN-β variant can be a naturally-occurring IFN-β having, e.g., an insertion, deletion or substitution of one or more amino acids relative to the wild-type IFN-β, i.e., a naturally occurring mutant or a polymorphic variant, or it can be a non-naturally occurring IFN-β.

“Isolated” (used interchangeably with “substantially pure”) when applied to polypeptides means a polypeptide which, by virtue of its origin or manipulation: (i) is present in a host cell as the expression product of a portion of an expression vector; (ii) is linked to a protein or other chemical moiety other than that to which it is linked in nature; or (iii) does not occur in nature, for example, a protein that is chemically manipulated by appending, or adding at least one hydrophobic moiety to the protein so that the protein is in a form not found in nature. By “isolated” it is further meant a protein that is: (i) synthesized chemically; or (ii) expressed in a host cell and purified away from associated and contaminating proteins. The term generally means a polypeptide that has been separated from other proteins and nucleic acids with which it naturally occurs. Preferably, the polypeptide is also separated from substances such as antibodies or gel matrices (polyacrylamide) which are used to purify it. “Isolated” (used interchangeably with “substantially pure”)—when applied to nucleic acids, refers to an RNA or DNA polynucleotide, portion of genomic polynucleotide, cDNA or synthetic polynucleotide which, by virtue of its origin or manipulation: (i) is not associated with all of a polynucleotide with which it is associated in nature (e.g., is present in a host cell as an expression vector or a portion thereof); or (ii) is linked to a nucleic acid or other chemical moiety other than that to which it is linked in nature; or (iii) does not Occur in nature. By “isolated” it is further meant a polynucleotide sequence that is: (i) amplified in vitro by, for example, polymerase chain reaction (PCR); (ii) synthesized chemically; (iii) produced recombinantly by cloning; or (iv) purified, as by cleavage and gel separation.

A nucleic acid is “operably linked” to another nucleic acid when it is placed into a functional relationship with another nucleic acid sequence. For example, DNA for a presequence or secretory leader (e.g., signal sequence or signal peptide) is operably linked to DNA encoding a polypeptide if the DNA is expressed as a preprotein that participates in the secretion of the polypeptide; a promoter or enhancer is operably linked to a coding sequence if it affects the transcription of the sequence; and a ribosome binding site is operably linked to a coding sequence if it is positioned so as to facilitate translation. Generally, “operably linked” means that the DNA sequences being linked are contiguous and, in the case of, e.g., a secretory leader, contiguous and in reading phase. Linking is accomplished by ligation at convenient restriction sites. If such sites do not exist, synthetic oligonucleotide adaptors or linkers can be used in accordance with conventional practice.

“Percent identity” or “percent similarity” refer to the sequence similarity between two polypeptides, molecules, or between two nucleic acids. When a position in both of the two compared sequences is occupied by the same base or amino acid monomer subunit, then the respective molecules are identical at that position. The percentage identity between two sequences is a function of the number of matching or identical positions shared by the two sequences divided by the number of positions compared×100. For instance, if 6 of 10 of the positions in two sequences are matched or are identical, then the two sequences are 60% homologous. By way of example, the DNA sequences CTGACT and CAGGTT share 50% homology (3 of the 6 total positions are matched). Generally, a comparison is made when two sequences are aligned to give maximum identity. Such alignment can be provided using, for instance, the method of Karlin and Altschul described in more detail below. When referring to a nucleic acid, “percent homology” and “percent identity” are used interchangeably, whereas when referring to a polypeptide, “percent homology” refers to the degree of similarity, where amino acids representing conserved substitutions of other amino acids are considered identical to these other amino acids. A “conservative substitution” of a residue in a reference sequence is a replacement with an amino acid that is physically or functionally similar to the corresponding reference residue, e.g., that have a similar size, shape, electric charge, chemical properties, including the ability to form covalent or hydrogen bonds, or the like. Particularly preferred conservative substitutions are those fulfilling the criteria defined for an “accepted point mutation” in Dayhoff et al., 5: Atlas of Protein Sequence and Structure, 5: Suppl. 3, chapter 22: 354-352, Nat. Biomed. Res. Foundation, Washington, D.C. (1978). The percent homology or identity of two amino acids sequences or two nucleic acid sequences can be determined using the alignment algorithm of Karlin and Altschul (Proc. Nat. Acad. Sci., USA 87: 2264 (1990) as modified in Karlin and Altschul (Proc. Nat. Acad. Sci., USA 90: 5873 (1993). Such an algorithm is incorporated into the NBLAST or XBLAST programs of Altschul et al., J. Mol. Biol. 215: 403 (1990). BLAST searches are performed with the NBLAST program, score=100, wordlength=12, to obtain nucleotide sequences homologous to a nucleic acid of the invention. BLAST protein searches are performed with the XBLAST program, score=50, wordlength=3, to obtain amino acid sequences homologous to a reference polypeptide. To obtain gapped alignments for comparisons, gapped BLAST is used as described in Altschul et al., Nucleic Acids Res., 25: 3389 (1997). When using BLAST and Gapped BLAST, the default parameters of the respective programs (XBLAST and NBLAST) are used. See http://www/ncbi.nlm.nih.gov.

An IFN-β therapeutic is said to have “therapeutic efficacy,” and an amount of the IFN-β therapeutic is said to be “therapeutically effective,” if administration of that amount of the IFN-β therapeutic is sufficient to cause a clinically significant improvement in a standard marker of renal function when administered to a subject (e.g., an animal model or human patient) having, or at risk of developing, glomerulonephritis or chronic renal failure. Such markers of renal function are well known in the medical literature and include, without being limited to, rates of increase in BUN levels, rates of increase in serum creatinine, static measurements of BUN, static measurements of serum creatinine, glomerular filtration rates (GFR), ratios of BUN/creatinine, serum concentrations of sodium (Na+), urine/plasma ratios for creatinine, urine/plasma ratios for urea, urine osmolality, daily urine output, and the like (see, for example, Brenner and Lazarus (1994), in Harrison's Principles of Internal Medicine, 13th edition, Isselbacher et al., eds., McGraw Hill Text, New York; Luke and Strom (1994), in Internal Medicine. 4th Edition, J. H. Stein, ed., Mosby-Year Book, Inc. St. Louis.). In a preferred embodiment, administration of a therapeutically effective amount of IFN-β therapeutic results in a decrease in proteinuria, glomerular cell proliferation or a decrease in the presence of inflammatory cells, e.g., CD8⁺ T cells and macrophages, in the glomeruli.

2. IFN-β Therapeutics

IFN-β therapeutics that can be used according to the invention include wild-type IFN-βs and biologically active variants thereof, e.g., naturally-occurring and non-naturally-occurring variants. The nucleotide and amino acid sequences of wild-type naturally-occurring human IFN-β are set forth in SEQ ID NOs: 1 and 2, respectively, which are identical to GenBank Accession Nos. M28622 and AAA36040, respectively. These IFNs are also described, e.g., in Seghal (1985) J. Interferon Res. 5:521. The full length human IFN-β protein is 187 amino acids long and the coding sequence of SEQ ID NO: 1 corresponds to nucleotides 76-639. The signal sequence corresponds to amino acids 1 to 21. The amino acid sequence of the mature form of this IFN-β corresponds to amino acids 22-187 (nucleotides 139-639 of SEQ ID NO: 1). The mature human IFN-β protein and nucleotide sequence encoding such are set forth as SEQ ID NOs: 4 and 3, respectively.

IFN-β produced in mammalian cells is glycosylated. Naturally-occurring wild-type IFN-β is glycosylated at residue 80 (Asn 80) of the mature polypeptide of SEQ ID NO: 4 or residue 101 (Asn 101 ) of the immature polypeptide of SEQ ID NO: 2.

IFN-β therapeutics also include non-human IFN-βs, e.g., from a vertebrate, such as a mammal, e.g., a non-human primate, bovine, ovine, porcine, equine, feline, canine, rat and mouse; or an avian or amphibian. IFN-β sequences from these species can be obtained from GenBank and/or publications, or can be determined from nucleic acids isolated by low stringency hybridization with an IFN-β gene from another species.

Variants of wild-type INF-β proteins include proteins having an amino acid sequence that is at least about 70%, 80%, 90%, 95%, 98% or 99% identical or homologous to a wild-type IFN-β, e.g., human IFN-β having SEQ ID NO: 2 or 4. Variants may have one or more amino acid substitutions, deletions or additions. For example, biologically active fragments of wild-type IFN-β proteins can be used. Such fragments may have 1, 2, 3, 5, 10 or up to 20 amino acids deleted, added or substituted at the C- or N-terminus of the protein. Variants may also have 1, 2, 3, 5, 10 or up to 20 amino acid substitutions, deletions or additions. Some variants may have less than about 50, 40, 30, 25, 20, 15, 10, 7, or 5 amino acid substitutions, deletions or additions. Substitutions can be with naturally occurring amino acids or with analogs thereof, e.g., D-stereoisomeric amino acids.

Also within the scope of the invention are IFN-β variants encoded by nucleic acids that hybridize under stringent conditions to a nucleic acid encoding a naturally-occurring IFN-β, e.g., represented by SEQ ID NOs: 1 or 3, or the complement thereof. Appropriate stringency conditions which promote DNA hybridization, for example, 6.0×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature of salt concentration may be held constant while the other variable is changed. In a preferred embodiment, a nucleic acid encoding an IFN-β variant will hybridize to one of SEQ ID NOs: 1 or 3 or complement thereof under moderately stringent conditions, for example at, and including a wash at, about 2.0×SSC and about 40° C. In a particularly preferred embodiment, a nucleic acid encoding an IFN-β variant will bind to one of SEQ-ID NOs: 1 or 3 or complement thereof under high stringency conditions, e.g., at, and including a wash at 0.2 SSC and about 65° C.

Exemplary modifications are conservative modifications, which have a minimal effect on the secondary and tertiary structure of the protein. Exemplary conservative substitutions include those described by Dayhoff in the Atlas of Protein Sequence and Structure 5 (1978), and by Argos in EMBO J., 8, 779-785 (1989). For example, amino acids belonging to one of the following groups represent conservative changes: ala, pro, gly, gin, asn, ser, thr; cys, ser, tyr, thr; val, ile, leu, met, ala, phe; lys, arg, his; and phe, tyr, trp, his.

Other modifications include the substitution of one amino acid for another amino acid that may not necessarily represent a conservative substitution. For example substitutions that essentially do not affect the three dimensional structure of IFN-β can be made. The three dimensional structure of non-glycosylated human IFN-β is described, e.g., in Radhakrishnan et al. (1996) Structure 4: 1453 and the three dimensional structure of glycosylated IFN-β is described, e.g., in Karpusas et al. (1997) PNAS 94:11813). Essentially, IFN-β comprises five helices: helix A, which consists of about amino acids 2-22 of SEQ ID NO: 4; helix B, which consists of about amino acids 51-71 of SEQ ID NO: 4; helix C, which consists of about amino acids 80-107 of SEQ ID NO: 4; helix D, which consists of about amino acids 118-136 of SEQ ID NO: 4 and helix E, which consists of about amino acids 139-162 of SEQ ID NO: 4 (Karpusas et al., supra). Helices A, B, C and E form a left-handed, type 2 four-helix bundle. There is a long overhand loop, the AB loop, that connects helices A and B and three shorter loops (named BC, CD and DE) that connects the rest of the helices (Karpusa et al., supra). Previous studies have shown that the N-terminal, C-terminal and the glycosylated C helix regions of the INF-beta molecule do not lie within the receptor binding site (see, WO 00/23472 and U.S. Ser. No. 09/832,659). Accordingly, mutations in these regions would not significantly adversely affect the biological activity of the IFN molecule. It has also been previously shown that mutations in helix C (amino acids 81, 82, 85, 86 and 89 of mature human IFN-β) results in a molecule having higher antiviral activity relative to the wild-type IFN-β (see, WO 00/23472 and U.S. Ser. No. 09/832,659). Similarly, it has been shown that mutants in the helix A (amino acids 2, 4, 5, 8 and 11 of mature human IFN-β) and CD loop (amino acids 110, 11, 113, 116 and 119) have a higher binding activity to the receptor and higher antiviral and anti-proliferative activities relative to the naturally occurring wild-type human IFN-β (see, WO 00/23472 and U.S. Ser. No. 09/832,659).

Other preferred modifications or substitutions eliminate sites for intermolecular crosslinking or incorrect disulfide bond formation. For example, IFN-β is known to have three cys residues, at wild-type positions 17, 31 and 141 of SEQ ID NO: 4. One IFN variant is an IFN in which the cys (C) at position 17 has been substituted with ser (S), as described, e.g., in U.S. Pat. No. 4,588,585. Other IFN-β variants include IFN-β variants having, e.g., one or more of ser (S) substituted for cys (C) at position 17 and val (V) at position 101 substituted with phe (F), trp (W), tyr (Y), or his (H), preferably phe (F), when numbered in accordance with wild type IFN-.β, having, e.g., SEQ ID NO: 4, such as described, e.g., in U.S. Pat. No. 6,127,332. Other preferred variants include polypeptides having the sequence of a wild-type IFN-.β, e.g., having SEQ ID NO: 4, wherein the val (V) at position 101, when numbered in accordance with wild type IFN-.β, is substituted with phe (F), tyr (Y), trp (W), his (I), or phe (F), also as described, e.g., in U.S. Pat. No. 6,127,332.

Other IFN-β variants are mature IFN-β molecules lacking the initiator methionine, e.g., methionine 1 of SEQ ID NO: 4. Exemplary IFN-β variants lack an initiator methionine and have at least one amino acid substitution, e.g., at position 17 of the mature form, as disclosed in U.S. Pat. No. 4,588,585.

IFN-β molecules can also be modified by replacing one or more amino acids with one or more derivatized amino acids, which are natural or nonnatural amino acid in which the normally occurring side chain or end group is modified by chemical reaction. Such modifications include, for example, gamma-carboxylation, beta-carboxylation, pegylation, sulfation, sulfonation, phosphorylation, amidization, esterification, N-acetylation, carbobenzylation, tosylation, and other modifications known in the art.

Other modifications include the use of amino acid analogs or derivatized amino acids wherein a side chain is lengthened or shortened while still providing a carboxyl, amino or other reactive precursor functional group for cyclization, as well as amino acid analogs having variant side chains with appropriate functional groups. For instance, the subject compound can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminopimelic acid, ornithine, or diaminobutyric acid. Other naturally occurring amino acid metabolites or precursors having side chains which are suitable herein will be recognized by those skilled in the art and are included in the scope of the present invention.

Other INF-β variants include reversed or retro peptide sequences. A “reversed” or “retro” peptide sequence refers to that part of an overall sequence of covalently-bonded amino acid residues (or analogs or mimetics thereof) wherein the normal carboxyl-to amino direction of peptide bond formation in the amino acid backbone has been reversed such that, reading in the conventional left-to-right direction, the amino portion of the peptide bond precedes (rather than follows) the carbonyl portion. See, generally, Goodman, M. and Chorev, M. Accounts of Chem. Res. 1979, 12, 423. The reversed orientation peptides described herein include (a) those wherein one or more amino-terminal residues are converted to a reversed (“rev”) orientation (thus yielding a second “carboxyl terminus” at the left-most portion of the molecule), and (b) those wherein one or more carboxyl-terminal residues are converted to a reversed (“rev”) orientation (yielding a second “amino terminus” at the right-most portion of the molecule). A peptide (amide) bond cannot be formed at the interface between a normal orientation residue and a reverse Orientation residue. Therefore, certain reversed polypeptides of the invention can be formed by utilizing an appropriate amino acid mimetic moiety to link the two adjacent portions of the sequences utilizing a reversed peptide (reversed amide) bond. In case (a) above, a central residue of a diketo compound may conveniently be utilized to link structures with two amide bonds to achieve a peptidomimetic structure. In case (b) above, a central residue of a diamino compound will likewise be useful to link structures with two amide bonds to form a peptidomimetic structure. The reversed direction of bonding in such polypeptides will generally, in addition, require inversion of the enantiomeric configuration of the reversed amino acid residues in order to maintain a spatial orientation of side chains that is similar to that of the non-reversed peptide. The configuration of amino acids in the reversed portion of the peptides is preferably (D), and the configuration of the non-reversed portion is preferably (L). Opposite or mixed configurations are acceptable when appropriate to optimize a binding activity. Modifications of polypeptides are further described, e.g., in U.S. Pat. No. 6,399,075.

IFN-β therapeutics also include IFN-β proteins and variants thereof (e.g., a mature protein) fused to a heterologous polypeptide. A heterologous polyeptide may be added, e.g., for the purpose of prolonging the half-life of the IFN-β protein or improving its production. Exemplary heterologous polypeptides include immunoglobulin (Ig) molecules or portions thereof, e.g., the constant domain of a light or heavy chain of an Ig molecule. In one embodiment, an IFN-β protein or variant thereof is fused or otherwise linked to all or part of the hinge and constant regions of an immunoglobulin light chain, heavy chain, or both. Thus, this invention features a molecule which includes: (1) an IFN-β protein moiety (i.e., an IFN-β or variant thereof), (2) a second peptide, e.g., one which increases solubility or in vivo life time of the IFN-β moiety, e.g., a member of the immunoglobulin super family or fragment or portion thereof, e.g., a portion or a fragment of IgG, e.g., the human IgGl heavy chain constant region, e.g., CH2, CH3, and hinge regions. Specifically, an “IFN-β/Ig fusion” is a protein comprising a biologically active IFN-β moiety linked to the N-terminus of an immunoglobulin chain. A species of IFN-β/Ig fusion is an “IFN-β/Fc fusion” which is a protein comprising an IFN-β moiety linked to at least a portion of the constant domain of an immunoglobulin. A preferred Fc fusion comprises an IFN-β moiety linked to a fragment of an antibody containing the C terminal domain of the heavy immunoglobulin chains.

Accordingly, in one embodiment, a fusion protein has the generic formula X-Y-Z, wherein X is a polypeptide having an amino acid sequence of IFN-β, or portion or variant thereof; Y is an optional linker moiety; and Z is a polypeptide comprising at least a portion of a polypeptide other than the interferon beta of moiety X. In other embodiments, the fusion protein has the formula Z-Y-X, in which the non-IFN-β polypeptide is fused to the N-terminal portion of the linker which is fused to the N-terminal portion of the IFN-β polypeptide or portion or variant thereof. Moiety Z can be a portion of a polypeptide that contains immunoglobulin-like domains. Examples of such other polypeptides include CD1, CD2, CD4, and members of class I and class II major histocompatability antigens. See U.S. Pat. No. 5,565,335 (Capon et al.) for examples of such polypeptides.

Moiety Z can include, for instance, a plurality of histidine residues or, preferably, the Fc region of an immunoglobulin, “Fc” defined herein as a fragment of an antibody containing the C terminal domain of the heavy immunoglobulin chains.

Moiety Y can be any linker that permits the IFN-β moiety to retain its biological activity. Moiety Y can be one amino acid long or at least two amino acids long. Y can also be from about 2 to about 5 amino acids; from about 3 to about 10 amino acid long or 10 or more amino acids. In a preferred embodiment, Y consists of or comprises GlyGlyGlylySer (SEQ ID NO: 6), which is encoded, e.g., by the nucleotide sequence GGCGGTGGTGGCAGC (SEQ ID NO: 5). Y can also consist of or comprise an enterokinase recognition site, e.g., AspAspAspAspLys (SEQ ID NO: 8), which is encoded by, e.g., GACGATGATGACAAG (SEQ ID NO: 7). In another embodiment, Y consists of or comprises SerSerGlyAspAspAspAspLys (SEQ ID NO: 10), which is encoded, e.g., by AGCTCCGGAGACGATGATGACAAG (SEQ ID NO: 9).

Moreover, the coupling between the IFN-β moiety (X) and the second, non-IFN-β moiety Z (e.g., an Fc region of an immunoglobulin) can also be effected by any chemical reaction that will bind the two molecules together so long as the X and Z moieties essentially retain their respective activities. This chemical linkage can include many chemical mechanisms such as covalent binding, affinity binding, intercalation, coordinate binding and complexation. Representative coupling agents (i.e., linkers “Y” in the generic formula) to develop covalent binding between the IFN-β moiety and Z moiety can include organic compounds such as thioesters, carbodiimides, succinimide esters, diisocyanates such as tolylene-2,6-diisocyanate, gluteraldehydes, diazobenzenes and hexamethylene diamines such as bis-(p-diazonium-benzoyl)ethylenediamine, bifunctional derivatives of imidoesters such as dimethyl adipimidate, and bis-active fluorine compounds such as 1,5-difluoro-2,4dinitrobenzene. This listing is not intended to be exhaustive of the various classes of chemical coupling agents known in the art Many of these are commercially available such as N-succinimidyl-3-(2-pyridyldithio) propionate (SPDP), 1-ethyl-3-(3-dimethylamino-propyl)carbodiimide hydrochloride (EDC); 4-succinimidyloxycarbonyl-alpha-methyl-alpha-(2-pyridyl-dithio)-toluene (SMPT: Pierce Chem. Co., Cat. #21558G).

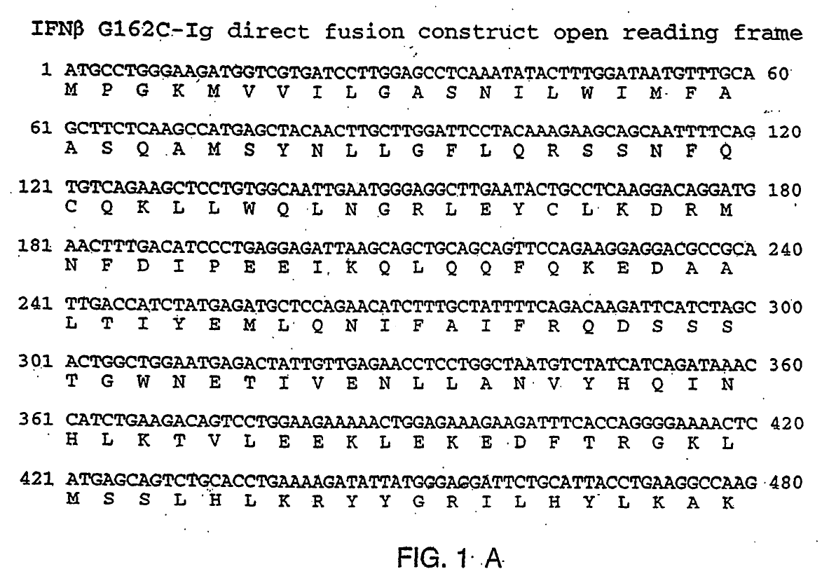

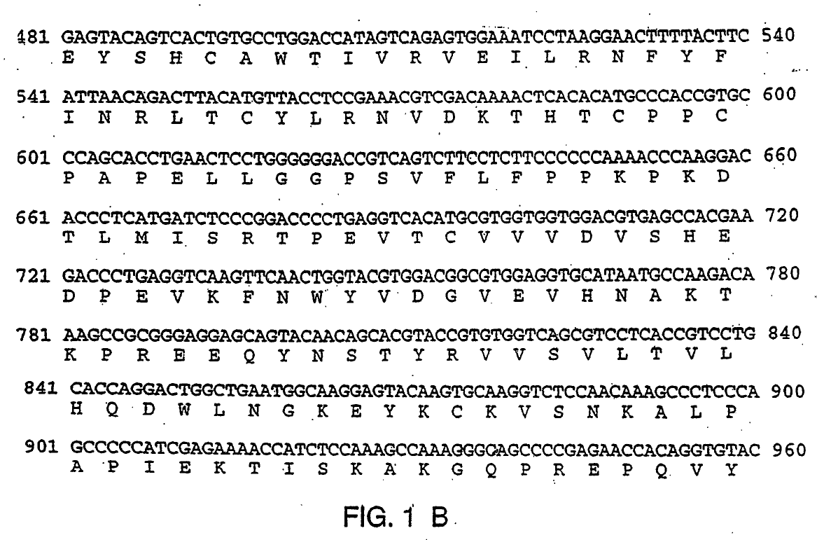

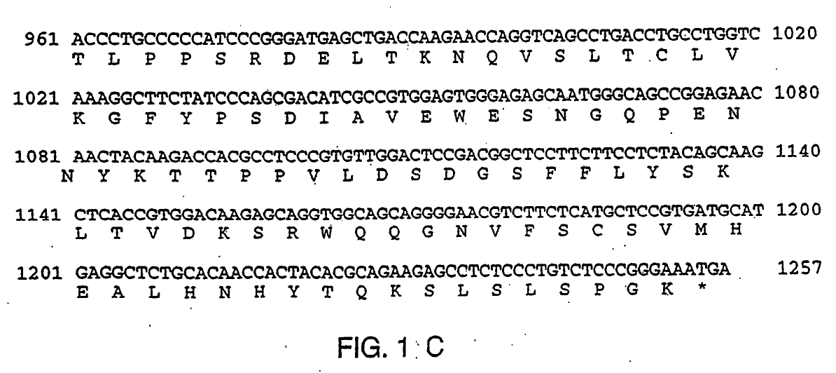

A preferred IFN-β/Ig fusion protein consists of or comprises SEQ ID NO: 12, which contains the full length mature form of human IFN-β, i.e., SEQ ID NO: 4, fused to human IgGlFc (ZL5107) (see WO 00/23472 and U.S. Ser. No. 09/832,659) (see FIG. 1). The corresponding nucleotide sequence is set forth in SEQ ID NO: 11. The DNA encoding human IFN-β ends at nucleotide triplet 568-570 (AAC encoding an arginine) and DNA encoding a human IgGl constant region starts at the triplet (GAC encoding an aspartic acid) beginning with nucleotide number 574 of SEQ ID NO: 11.

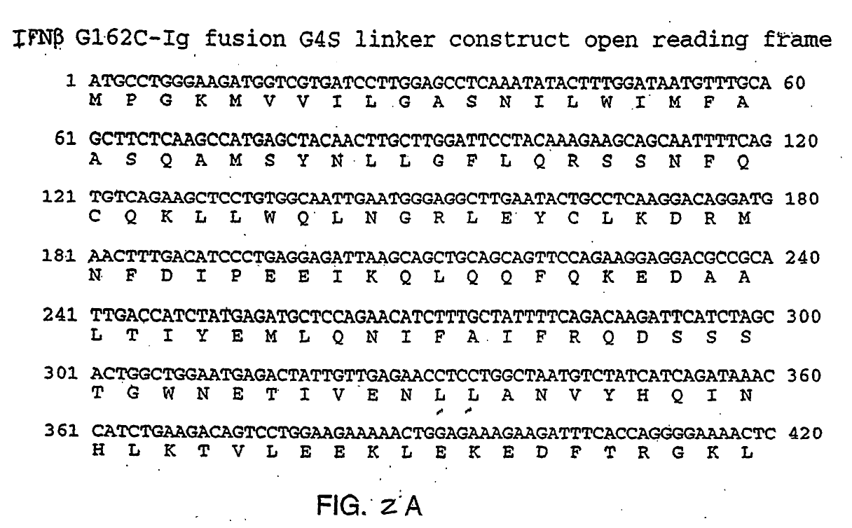

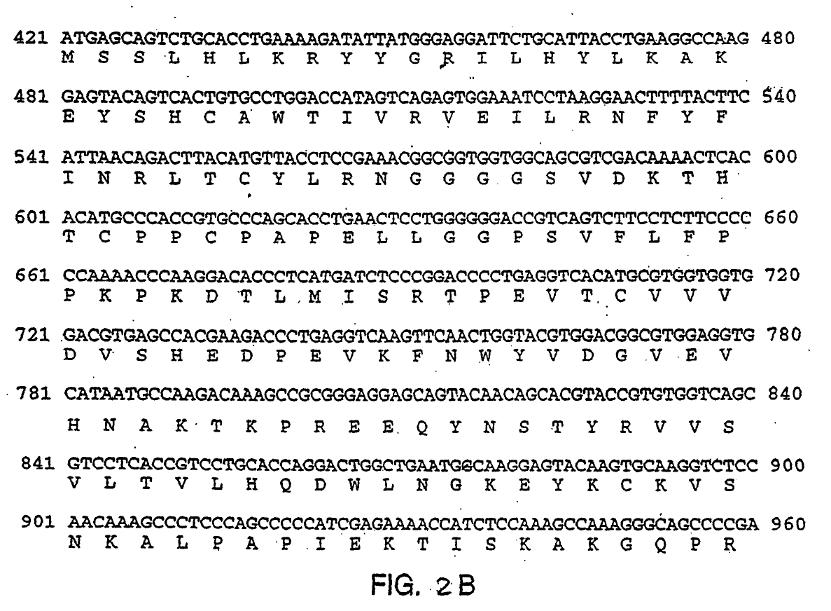

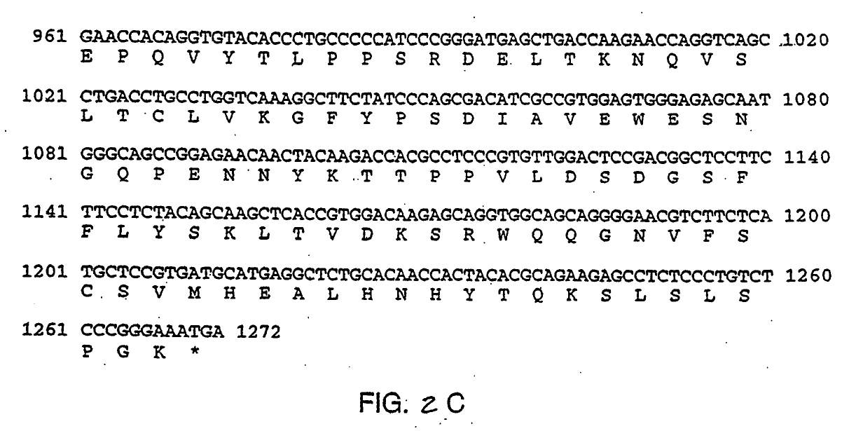

Another preferred IFN-β/Ig fusion protein is set forth in SEQ ID NO: 14 and encoded by SEQ ID NO: 13 (see WO 00/23472 and U.S. Ser. No. 09/832,659) (see FIG. 2). This latter fusion protein consists of human IFN-β linked to the G4S linker that is itself linked to human IgGlFc (ZL6206). The G4S linker (encoded by nucleotides 571 to 585 of SEQ ID NO: 7) consists of the amino acid sequence GGGGS (SEQ ID NO: 9). Methods for producing these proteins are described in WO 00/23472 and U.S. Ser. No. 09/832,659.

In a preferred embodiment, the IFN-β polypeptide is fused via its C-terminus to at least a portion of the Fc region of an immunoglobulin. The IFN-β forms the amino-terminal portion, and the Fc region forms the carboxy terminal portion. In these fusion proteins, the Fc region is preferably limited to the constant domain hinge region and the CH2 and CH3 domains. The Fc region in these fusions can also be limited to a portion of the hinge region, the portion being capable of forming intermolecular disulfide bridges, and the CH2 and CH3 domains, or functional equivalents thereof. These constant regions may be derived from any mammalian source (preferably human) and may be derived from any appropriate class and/or isotype, including IgA, IgD, IgM, IgE and IgG1, IgG2, IgG3 and IgG4.

Recombinant nucleic acid molecules which encode the Ig fusions may be obtained by any method known in the art (Maniatis et al., 1982, Molecular Cloning; A Laboratory Manual, Cold Spring Harbor Laboratory, Cold Spring Harbor, N.Y.) or obtained from publicly available clones. Methods for the preparation of genes which encode the heavy or light chain constant regions of immunoglobulins are taught for example, by Robinson, R. et al., PCT Application, Publication No. WO87/02671. The cDNA sequence encoding the interferon molecule or fragment may be directly joined to the cDNA encoding the heavy Ig contant regions or may be joined via a linker sequence. In further embodiments of the invention, a recombinant vector system may be created to accommodate sequences encoding interferon beta in the correct reading frame with a synthetic hinge region. Additionally, it may be desirable to include, as part of the recombinant vector system, nucleic acids corresponding to the 3′ flanking region of an immunoglobulin gene including RNA cleavage/polyadenylation sites and downstream sequences. Furthermore, it may be desirable to engineer a signal sequence upstream of the immunoglobulin fusion protein-encoding sequences to facilitate the secretion of the fused molecule from a cell transformed with the recombinant vector.

The present invention provides for dimeric fusion molecules as well as monomeric or multimeric molecules comprising fusion proteins. Such multimers may be generated by using those Fc regions, or portions thereof, of Ig molecules which are usually multivalent such as IgM pentamers or IgA dimers. It is understood that a J chain polypeptide may be needed to form and stabilize IgM pentamers and IgA dimers. Alternatively, multimers of IFN-β fusion proteins may be formed using a protein with an affinity for the Fc region of Ig molecules, such as Protein A. For instance, a plurality of IFN-β/immunoglobulin fusion proteins may be bound to Protein A-agarose beads.

These polyvalent forms are useful since they possess multiple interferon beta receptor binding sites. For example, a bivalent soluble IFN-β may consist of two tandem repeats of amino acids 1 to 166 of SEQ ID NO: 4 (or those encoded by nucleic acids numbered 1 to 498 of SEQ. ID. NO: 3) (moiety X in the generic formula) separated by a linker region (moiety Y), the repeats bound to at least a portion of an immunoglobulin constant domain (moiety Z). Alternate polyvalent forms may also be constructed, for example, by chemically coupling IFN-β/Ig fusions to any clinically acceptable carrier molecule, a polymer selected from the group consisting of Ficoll, polyethylene glycol or dextran using conventional coupling techniques. Alternatively, IFN-βmay be chemically coupled to biotin, and the biotin-interferon beta Fe conjugate then allowed to bind to avidin, resulting in tetravalent avidin/biotin/interferon beta molecules. IFN-β/Ig fusions may also be covalently coupled to dinitrophenol (DNP) or trinitrophenol (TNP) and the resulting conjugate precipitated with anti-DNP or anti-TNP-IgM, to form decameric conjugates with a valency of 10 for interferon beta receptor binding sites

Derivatives of proteins of the invention also include various structural forms of the primary protein which retain biological activity. Due to the presence of ionizable amino and carboxyl groups, for example, IFN-β proteins and variants thereof may be in the form of acidic or basic salts, or may be in neutral form. Individual amino acid residues may also be modified by oxidation or reduction. Further, the primary amino acid structure (including the N- and/or C-terminal ends) or the glycan of the IFN-β may be modified (“derivatized”) by forming covalent or aggregative conjugates with other chemical moieties, such as glycosyl groups, polyalkylene glycol polymers such as polyethylene glycol, lipids, phosphate, acetyl groups and the like, or by creating amino acid sequence mutants.

Other derivatives of interferon beta/Ig include covalent or aggregative conjugates of interferon beta or its fragments with other proteins or polypeptides, such as by synthesis in recombinant culture as additional N-termini, or C-termini. For example, the conjugated peptide may be a signal (or leader) polypeptide sequence at the N-terminal region of the protein which co-translationally or post-translationally directs transfer of the protein from its site of synthesis to its site of function inside or outside of the cell membrane or wall (e.g., the yeast alpha-factor leader). For example, the signal peptide can be that of IFN-β, i.e., amino acids 1-21 of SEQ ID NO: 2, corresponding to nucleotides 1-138 of SEQ ID NO: 1. The signal peptide can also be that of VCAM, i.e., amino acids 1-24 of SEQ ID NO: 12, which is encoded by nucleotides 1-72 of SEQ ID NO: 11.

A heterologous polypeptide (e.g., peptide) or other molecule may also be used as a label or for helping in the purification of the IFN-β therapeutic. Such peptides are well known in the art. For example, the polynucleotide of the present invention may be fused in frame to a marker sequence, also referred to herein as “Tag sequence” encoding a “Tag peptide,” which allows for marking and/or purification of the polypeptide of the present invention. In a preferred embodiment, the marker sequence is a hexahistidine tag, e.g., supplied by a PQE-9 vector. Numerous other Tag peptides are available commercially. Other frequently used Tags include myc-epitopes (e.g., see Ellison et al. (1991) J Biol Chem 266:21150-21157), which includes a 10-residue sequence from c-myc, the pFLAG system (International Biotechnologies, Inc.), the pEZZ-protein A system (Pharmacia, NJ), and a 16 amino acid portion of the Haemophilus influenza hemagglutinin protein. Furthermore, any polypeptide can be used as a Tag so long as a reagent, e.g., an antibody interacting specifically with the Tag polypeptide is available or can be prepared or identified.

In one embodiment, an IFN-β protein or variant thereof is fused at the N- or C-terminus with one of the following peptides: HisHisHis HisHisHis (SEQ ID NO: 16), which may be encoded by the nucleotide sequence CATCATCATCATCATCAT (SEQ ID NO: 15); SerGlyGlyHisHisHisHisHisHis (SEQ ID NO: 18), which may be encoded by the nucleotide sequence TCCGGGGGCCATCATCATCATCATCAT (SEQ ID NO: 15) and SerGlyGlyHisHisHisrHisHisSerSerGlyAspAspAspAspLys (SEQ ID NO: 20), which may be encoded by the nucleotide sequence TCCGGGGGCCATCATCATCATCATCATAGCTCCGGAGACGATGATGACAAG (SEQ ID NO: 19).

The amino acid sequence of interferon beta can also be linked to the peptide AspTyrLysAspAspAspAspLys (DYKDDDDK) (SEQ ID NO: 21) (Hopp et al., Bio/Technology 6:1204,1988). The latter sequence is highly antigenic and provides an epitope reversibly bound by a specific monoclonal antibody, enabling rapid assay and facile purification of expressed recombinant protein. This sequence is also specifically cleaved by bovine mucosal enterokinase at the residue immediately following the Asp-Lys pairing.

In another embodiment, an IFN-β therapeutic comprises an IFN-β protein or variant thereof fused to an albumin protein, variant or portion thereof. Such a fusion protein can be created as described in, e.g., WO 01/77137.

IFN-β therapeutics may also include a molecule that is not a polypeptide. For example, an IFN-β protein or variant thereof can be linked covalently or not covalently to a polymer, eg., a biodegradable polymer. For example, an IFN-β protein or variant thereof can be pegylated, e.g., linked to polyethylene glycol (PEG), as described in WO 00/23114.

Within the broad scope of the present invention, a single polymer molecule may be employed for conjugation with an IFN-β, although it is also contemplated that more than one polymer molecule can be attached as well. It will be recognized that the conjugating polymer may utilize any groups, moieties, or other conjugated species, as appropriate to the end use application. By way of example, it may be useful in some applications to covalently bond to the polymer a functional moiety imparting UV-degradation resistance, or antioxidation, or other properties or characteristics to the polymer. As a further example, it may be advantageous in some applications to functionalize the polymer to render it reactive or cross-linkable in character, to enhance various properties or characterisics of the overall conjugated material. Accordingly, the polymer may contain any functionality, repeating groups, linkages, or other constitutent structures which do not preclude the efficacy of the conjugated IFN-β composition for its intended purpose.

The IFN-β is conjugated most preferably via a terminal reactive group on the polymer although conjugations can also be branched from the non-terminal reactive groups. The polymer with the reactive group(s) is designated herein as “activated polymer.” The reactive group selectively reacts with free amino or other reactive groups on the protein. The activated polymer(s) are reacted so that attachment may occur at any available IFN-β amino group such as the alpha amino groups or the epsilon-amino groups of lysines. Free carboxylic groups, suitably activated carbonyl groups, hydroxyl, guanidyl, oxidized carbohydrate moieties and mercapto groups of the IFN-β (if available) can also be used as attachment sites.

Although the polymer may be attached anywhere on the IFN-β molecule or variant thereof or other amino acid linked directly or indirectly to the IFN-β molecule, the most preferred site for polymer coupling is the N-terminus of the IFN-β molecule. Secondary site(s) are at or near the C-terminus and through sugar moieties. Thus, the invention contemplates as its most preferred embodiments: (i) N-terminally coupled polymer conjugates of IFN-β or variant thereof; (ii) C-terminally coupled polymer conjugates of IFN-β or variant thereof; (iii) sugar-coupled conjugates of polymer conjugates; (iv) as well as N-, C- and sugar-coupled polymer conjugates of IFN-β proteins or variants thereof.

Generally from about 1.0 to about 10 moles of activated polymer per mole of protein, depending on protein concentration, is employed. The final amount is a balance between maximizing the extent of the reaction while minimizing non-specific modifications of the product and, at the same time, defining chemistries that will maintain optimum activity, while at the same time optimizing, if possible, the half-life of the protein. Preferably, at least about 50% of the biological activity of the protein is retained, and most preferably 100% is retained.

The reactions may take place by any suitable method used for reacting biologically active materials with inert polymers, preferably at about pH 5-7 if the reactive groups are on the alpha amino group at the N-terminus. Generally the process involves preparing an activated polymer (that may have at least one terminal hydroxyl group) and thereafter reacting the protein with the activated polymer to produce the soluble protein suitable for formulation. The above modification reaction can be performed by several methods, which may involve one or more steps.

As mentioned above, the most preferred embodiments of the invention utilize the N-terminal end of IFN-β as the linkage to the polymer. Suitable methods are available to selectively obtain an N-terminally modified IFN-β. One method is exemplified by a reductive alkylation method which exploits differential reactivity of different types of primary amino groups (the epsilon amino groups on the lysine versus the amino groups on the N-terminal methionine) available for derivatization on IFN-β. Under the appropriate selection conditions, substantially selective derivatization of IFN-β at its N-terminus with a carbonyl group containing polymer can be achieved. The reaction is performed at a pH which allows one to take advantage of the pKa differences between the epsilon-amino groups of the lysine residues and that of the alpha-amino group of the N-terminal residue of IFN-β. This type of chemistry is well known to persons with ordinary skill in the art.

For example, a reaction scheme can be used in which this selectivity is maintained by performing reactions at low pH (generally 5-6) under conditions where a PEG-aldehyde polymer is reacted with IFN-β in the presence of sodium cyanoborohydride. After purification of the PEG-IFN-β and analysis with SDS-PAGE, MALDI mass spectrometry and peptide sequencing/mapping, this resulted in an IFN-β whose N-terminus is specifically targeted by the PEG moiety.

The crystal structure of IFN-β indicates that the N- and C-termini are located close to each other (see Karpusas et al., 1997, Proc. Natl. Acad. Sci. 94: 11813-11818). Thus, modifications of the C-terminal end of IFN-β should also have minimal effect on activity. While there is no simple chemical strategy for targeting a polyalkylene glycol polymer such as PEG to the C-terminus, it would be straightforward to genetically engineer a site that can be used to target the polymer moiety. For example, incorporation of a Cys at a site that is at or near the C-terminus would allow specific modification using a maleimide, vinylsulfone or haloacetate-activated polyalkylene glycol (e.g., PEG). These derivatives can be used specifically for modification of the engineered cysteines due to the high selectively of these reagents for Cys. Other strategies such as incorporation of a histidine tag which can be targeted (Fancy et al., (1996) Chem. & Biol. 3: 551) or an additional glycosylation site, represent other alternatives for modifying the C-terminus of IFN-β.

The glycan on certain IFN-β is also in a position that would allow further modification without altering activity. Methods for targeting sugars as sites for chemical modification are also well known and therefore it is likely that a polyalkylene glycol polymer can be added directly and specifically to sugars on IFN-β that have been activated through oxidation. For example, a polyethyleneglycol-hydrazide can be generated which forms relatively stable hydrazone linkages by condensation with aldehydes and ketones. This property has been used for modification of proteins through oxidized oligosaccharide linkages. See Andresz, H. et al., (1978), Makromol. Chem. 179: 301. In particular, treatment of PEG-carboxymethyl hydrazide with nitrite produces PEG-carboxymethyl azide which is an electrophilically active group reactive toward amino groups. This reaction can be used to prepare polyalkylene glycol-modified proteins as well. See, U.S. Pat. Nos. 4,101,380 and 4,179,337.

Thiol linker-mediated chemistry can further facilitate cross-linking of proteins. This can be performed, e.g., by generating reactive aldehydes on carbohydrate moieties with sodium periodate, forming cystamine conjugates through the aldehydes and inducing cross-linking via the thiol groups on the cystamines (see Pepinsky, B. et al., (1991), J. Biol. Chem., 266: 18244-18249 and Chen, L. L. et al., (1991) J. Biol. Chem., 266: 18237-18243). Accordingly, this type of chemistry is expected to be appropriate for modification with polyalkylene glycol polymers where a linker is incorporated into the sugar and the polyalkylene glycol polymer is attached to the linker. While aminothiol or hydrazine-containing linkers will allow for addition of a single polymer group, the structure of the linker can be varied so that multiple polymers are added and/or that the spatial orientation of the polymer with respect to the IFN-β is changed.

Exemplary polymers include water soluble polymer such as a polyalkylene glycol polymer. A non-limiting list of such polymers include other polyalkylene oxide homopolymers such as polypropylene glycols, polyoxyethylenated polyols, copolymers thereof and block copolymers thereof. Other examples of suitable water-soluble and non-peptidic polymer backbones include poly(oxyethylated polyol), poly(olefinic alcohol), poly(vinylpyrrolidone), poly(hydroxypropylmethacrylamide), poly(α-hydroxy acid), poly(vinyl alcohol), polyphosphazene, polyoxazoline, poly(N-acryloylmorpholine) and copolymers, terpolymers, and mixtures thereof. In one embodiment, the polymer backbone is poly(ethylene glycol) or monomethoxy polyethylene glycol (mPEG) having an average molecular weight from about 200 Da to about 400,000 Da. It should be understood that other related polymers are also suitable for use in the practice of this invention and that the use of the term PEG or poly(ethylene glycol) is intended to be inclusive and not exclusive in this respect. The term PEG includes poly(ethylene glycol) in any of its forms, including alkoxy PEG, difunctional PEG, multi-armed PEG, forked PEG, branched PEG, pendent PEG, or PEG with degradable linkages therein.

In one embodiment, polyalkylene glycol residues of C1-C4 alkyl polyalkylene glycols, preferably polyethylene glycol (PEG), or poly(oxy)alkylene glycol residues of such glycols are incorporated in the polymer systems of interest. Thus, the polymer to which the protein is attached can be a homopolymer of polyethylene glycol (PEG) or is a polyoxyethylated polyol, provided in all cases that the polymer is soluble in water at room temperature. Non-limiting examples of such polymers include polyalkylene oxide homopolymers such as PEG or polypropylene glycols, polyoxyethylenated glycols, copolymers thereof and block copolymers thereof, provided that the water solubility of the block copolymer is maintained. Examples of polyoxyethylated polyols include, for example, polyoxyethylated glycerol, polyoxyethylated sorbitol, polyoxyethylated glucose, or the like. The glycerol backbone of polyoxyethylated glycerol is the same backbone occurring naturally in, for example, animals and humans in mono-, di-, and triglycerides. Therefore, this branching would not necessarily be seen as a foreign agent in the body.

As an alternative to polyalkylene oxides, dextran, polyvinyl pyrrolidones, polyacrylamides, polyvinyl alcohols, carbohydrate-based polymers and the like may be used. Those of ordinary skill in the art will recognize that the foregoing list is merely illustrative and that all polymer materials having the qualities described herein are contemplated.

The polymer need not have any particular molecular weight, but it is preferred that the molecular weight be between about 300 and 100,000, more preferably between 10,000 and 40,000. In particular, sizes of 20,000 or more are best at preventing protein loss due to filtration in the kidneys.

Polyalkylene glycol derivatization has a number of advantageous properties in the formulation of polymer-IFN-β conjugates in the practice of the present invention, as associated with the following properties of polyalkylene glycol derivatives: improvement of aqueous solubility, while at the same time eliciting no antigenic or immunogenic response; high degrees of biocompatibility; absence of in vivo biodegradation of the polyalkylene glycol derivatives; and ease of excretion by living organisms.

Moreover, in another aspect of the invention, one can utilize IFN-β covalently bonded to the polymer component in which the nature of the conjugation involves cleavable covalent chemical bonds. This allows for control in terms of the time course over which the polymer may be cleaved from the IFN-β. This covalent bond between the IFN-β drug and the polymer may be cleaved by chemical or enzymatic reaction. The polymer-IFN-β product retains an acceptable amount of activity. Concurrently, portions of polyethylene glycol are present in the conjugating polymer to endow the polymer-IFN-β conjugate with high aqueous solubility and prolonged blood circulation capability. As a result of these improved characteristics the invention contemplates parenteral, nasal, and oral delivery of both the active polymer-IFN-β species and, following hydrolytic cleavage, bioavailability of the IFN-β per se, in in vivo applications.

The reaction of the polymer with the IFN-β to obtain conjugates, e.g., N-terminal conjugated products, can be readily carried out using a wide variety of reaction schemes. The activity and stability of the IFN-β conjugates can be varied in several ways, by using a polymer of different molecular size. Solubilities of the conjugates can be varied by changing the proportion and size of the polyethylene glycol fragment incorporated in the polymer composition.

In one embodiment, conjugates according to the present invention are prepared by reacting a protein with an activated polyaklylene glycol compound (PCG). For example, IFN can be reacted with a PEG-aldehyde in the presence of a reducing agent (e.g., sodium cyanoborohydride) via reductive alkylation to produce a PEG-protein conjugate, attached via an amine linkage. See, e.g., European Patent 0154316 B1 and International Patent Application No. PCT/US03/01559.

In certain embodiments of the invention, human IFN-β is PEGylated with the following activated polyalkylene glycols: 20 kDa mPEG-O-2-methylpropionaldehyde, 20 kDa mPEG-O-p-metylphenyl-O-2-methylpropionaldehyde, 20 kDa mPEG-O-m-methylphenyl-O-2-methylpropionaldehyde, 20 kDa mPEG-O-p-phenylacetaldehyde, 20 kDa mPEG-O-p-phenylpropionaldehyde, and 20 kDa mPEG-O-m-phenylacetaldehyde to obtain 20 kDa mPEG-O-2-methylpropionaldehyde-modified IFN-β, 20 kDa mPEG-O-p-methylphenyl-O-2-methylpropionaldehyde-modified IFN-β, 20 kDa mPEG-O-m-methylphenyl-O-2-methylpropionaldehyde-modified IFN-β, 20 kDa mPEG-O-p-phenylacetaldehyde-modified IFN-β, 20 kDa mPEG-O-p-phenylpropionaldehyde-modified IFN-β, and 20 kDa mPEG-O-m-phenylacetaldehyde-modified IFN-β, respectively. A detailed description of the preparation and characterization of human IFN-β modified with 20 kDa mPEG-O-2-methylpropionaldehyde and 20 kDa mPEG-O-p-phenylacetaldehyde is set forth below and is also provided in International Patent Application No. PCT/US03/01559.

In one embodiment, a pegylated IFN-β is prepared as follows. IFN-β, e.g., IFN-β-1a bulk intermediate (a clinical batch of bulk drug that passed all tests for use in humans) at 250 μg/ml in 100 mM sodium phosphate pH 7.2, 200 mM NaCl is diluted with an equal volume of 100 mM MES pH 5.0, and the pH was adjusted to 5.0 with HCl. The sample is loaded onto an SP-Sepharose® FF column (Pharmacia, Piscataway, N.J.) at 6 mg IFN-β/ml resin. The column is washed with 5 mM sodium phosphate pH 5.5, 75 mM NaCl, and the product is eluted with 30 mM sodium phosphate pH 6.0, 600 mM NaCl. Elution fractions can be analyzed for their absorbance values at 280 nm and the concentration of interferon in the samples estimated from the absorbance using an extinction coefficient of 1.51 for a 1 mg/ml solution.

To a 1 mg/ml solution of the IFN-β from the SP eluate, 0.5 M sodium phosphate pH 6.0 is added to 50 mM, sodium cyanoborohydride (Aldrich, Milwaukee, Wis.) is added to 5 mM, and 20K PEG aldehyde (Shearwater Polymers, Huntsville, Ala.) is added to 5 mg/ml. The sample is incubated at room temperature for 20 hours. The pegylated interferon is purified from reaction products by sequential chromatography steps on a Superose® 6 FPLC sizing column (Pharmacia) with 5 mM sodium phosphate pH 5.5, 150 mM NaCl as the mobile phase and SP-Sepharose® FF. The sizing column results in base line separation of modified and unmodified IFN-β. The PEG-interferon beta-containing elution pool from gel filtration is diluted 1:1 with water and loaded at 2 mg interferon beta/ml resin onto an SP-Sepharose® column. The column is washed with 5 mM sodium phosphate pH 5.5, 75 mM NaCl and then the pegylated interferon beta is eluted from the column with 5 mM sodium phosphate pH 5.5, 800 mM NaCl. Elution fractions are analyzed for protein content by absorbance at 280 nm. The pegylated interferon concentration is reported in interferon equivalents as the PEG moiety did not contribute to absorbance at 280 nm. These method and characterization of the pegylated IFN-β obtained are further described in WO 00/23114. PEG conjugation of IFN-β does not appear to alter its antiviral activity. In addition, the specific activity of pegylated IFN-β was found to be much greater (about 10 times) than that of the non-pegylated IFN-β (WO 00/23114).

IFN-β can also be pegylated with a 5K PEG-aldehyde moiety that can be purchased, e.g., from Fluka, Inc. (Cat. No. 75936, Ronkonkoman, N.Y.) following the same protocol as described above for the 20K PEG aldehyde.

A 20 kDa mPEG-O-2-methylpropionaldehyde-modified IFN-β can be prepared as follows. 10 mL of IFN-β-1a bulk intermediate (a clinical batch of bulk drug that passed all tests for use in humans) at 250 μg/mL in 100 mM sodium phosphate pH 7.2, 200 mM NaCl is diluted with 12 mL of 165 mM MES pH 5.0 and 50 μL of 5 N HCl. The sample is loaded onto a 300 μL SP-Sepharose FF column (Pharmacia). The column is washed with 3×300 μL of 5 mM sodium phosphate pH 5.5, 75 mM NaCl, and the protein is eluted with 5 mM sodium phosphate pH 5.5, 600 MM NaCl. Elution fractions are analyzed for their absorbance at 280 nm and the concentration of IFN-β in the samples estimated using an extinction coefficient of 1.51 for a 1 mg/mL solution. The peak fractions are pooled to give an IFN-β concentration of 3.66 mg/mL, which is subsequently diluted to 1.2 mg/mL with water.

To 0.8 mL of the IFN-β from the diluted SP-Sepharose eluate pool, 0.5 M sodium phosphate pH 6.0 is added to 50 mM, sodium cyanoborohdride (Aldrich) is added to 5 mM, and 20 kDa mPEG-O-2-methylpropionaldehyde is added to 5 mg/mL. The sample is incubated at room temperature for 16 h in the dark. The PEGylated IFN-β is purified from the reaction mixture on a 0.5 mL SP-Sepharose FF column as follows: 0.6 mL of the reaction mixture is diluted with 2.4 mL 20 mM MES pH 5.0, and loaded on to the SP-Sepharose column. The column is washed with sodium phosphate pH 5.5, 75 mM NaCl and then the PEGylated IFN-β is eluted from the column with 25 mM MES pH 6.4, 400 mM NaCl. The PEGylated IFN-β is further purified on a Superose 6 HR 10/30 FPLC sizing column with 5 mM sodium phosphate pH 5.5, 150 mM NaCl as the mobile phase. The sizing column (25 mL) is run at 20 mL/h and 0.5 mL fractions are collected. The elution fractions are analyzed for protein content by absorbance at 280 nm, pooled, and the protein concentration of the pool determined. The PEGylated IFN-β concentration is reported in IFN equivalents as the PEG moiety does not contribute to absorbance at 280 nm. Samples of the pool are removed for analysis, and the remainder can be diluted to 30 μg/mL with HSA-containing formulation buffer, aliquoted at 0.25 mL/vial, and stored at −70° C.

20 kDa mPEG-O-p-phenylacetaldehyde-modified IFN-β can be prepared as follows. 20 mL of ®IFN-β bulk intermediate (a clinical batch of bulk drug that passed all tests for use in humans) at 250 μg/mL in 100 mM sodium phosphate pH 7.2, 200 mM NaCl is diluted with 24 mL of 165 mM MES pH 5.0, 100 μL of 5 N HCl, and 24 mL water. The sample is loaded onto a 600 μL SP-Sepharose FF column (Pharmacia). The column is washed with 2×900 μL of 5 mM sodium phosphate pH 5.5, 75 mM NaCl, and the protein is eluted with 5 mM sodium phosphate pH 5.5, 600 mM NaCl. Elution fractions are analyzed for their absorbance at 280 nm and the concentration of IFN-β in the samples was estimated using an extinction coefficient of 1.51 for a 1 mg/mL solution. The peak fractions are pooled to give an IFN-β concentration of 2.3 mg/mL. To 1.2 mL of the IFN-β-1a from the SP-Sepharose eluate pool, 0.5 M sodium phosphate pH 6.0 is added to 50 mM, sodium cyanoborohdride (Aldrich) is added to 5 mM, and 20 kDa mPEG-O-p-phenylacetaldehyde, is added to 10 mg/mL. The sample is incubated at room temperature for 18 h in the dark. The PEGylated IFN-β can be purified from the reaction mixture on a 0.75 mL SP-Sepharose FF column as follows: 1.5 mL of reaction mixture is diluted with 7.5 mL 20 mM MES pH 5.0, 7.5 mL water, and 5 μL 5 N HCl, and loaded onto the SP-Sepharose column. The column is washed with sodium phosphate pH 5.5, 75 mM NaCl and then the PEGylated IFN-β is eluted from the column with 20 mM MES pH 6.0, 600 mM NaCl. The PEGylated IFN-β is further purified-on a Superose 6 HR 10/30 FPLC sizing column with 5 mM sodium phosphate pH 5.5, 150 mM NaCl as the mobile phase. The sizing column (25 mL) is run at 20 mL/h and 0.5 mL fractions are collected. The elution fractions are analyzed for protein content by absorbance at 280 nm, pooled, and the protein concentration of the pool determined. The PEGylated IFN-β concentration is reported in IFN equivalents after adjusting for the contribution of the PEG (20 kDa mPEG-O-p-phenylacetaldehyde has an extinction coefficient at 280 nm of 0.5 for a 1 mg/mL solution) to the absorbance at 280 nm using an extinction coefficient of 2 for a 1 mg/mL solution of the PEGylated IFN-β. Samples of the pool can be removed for analysis, and the remainder can be diluted to 30 μg/mL with HSA-containing formulation buffer, aliquoted at 0.25 mL/vial, and stored at −70° C.

Glycosylated IFN-β coupled to a non-naturally occurring polymer can be used in the methods of the invention. The polymer may comprise a polyalkylene glycol moiety. The polyalkylene moiety may be coupled to the interferon-beta by way of a group selected from an aldehyde group, a maleimide group, a vinylsulfone group, a haloacetate group, plurality of histidine residues, a hydrazine group and an aminothiol group. IFN-β may be coupled to a polyethylene glycol moiety, wherein the IFN-β is coupled to the polyethylene glycol moiety by a labile bond, wherein the labile bond is cleavable by biochemical hydrolysis and/or proteolysis. The polymer may have a molecular weight of from about 5 to about 40 kilodaltons. Another IFN-β that may be used is a physiologically active interferon-beta composition comprising a physiologically active glycosylated interferon-beta N-terminally coupled to a polymer comprising a polyalkylene glycol moiety, wherein the physiologically active interferon-beta and the polyalkylene glycol moiety are arranged such that the physiologically active interferon-beta in the physiologically active interferon-beta composition has substantially similar activity relative to physiologically active interferon-beta lacking said moiety, when measured by an antiviral assay.

Heterologous polypeptides or other molecules can be covalently or non-covalently linked to an IFN-β protein or variant thereof. “Covalently coupled” means that the different moieties of the invention are either directly covalently bonded to one another, or else are indirectly covalently joined to one another through an intervening moiety or moieties, such as a bridge, spacer, or linkage moiety or moieties. The intervening moiety or moieties are called a “coupling group.” The term “conjugated” is used interchangeably with “covalently coupled.”

IFN-βs for use in the invention can be glycosylated or non-glycosylated (or unglycosylated). Non-glycosylated IFN-βs can be produced, e.g., in a prokaryotic host cell. INF-β proteins or variants thereof can also be modified by attaching polysaccharides that are not normally present on IFN-βs.

3. Methods of Producing INF-β Therapeutics

The IFN-β therapeutics of the present invention can be produced by any suitable methods, such as methods including constructing a nucleic acid encoding an IFN-β therapeutic and expressing this nucleic acid in a suitable transformed host. This method will produce recombinant IFN-β therapeutics. IFN-β therapeutics may also be produced by chemical synthesis or a combination of chemical synthesis and recombinant DNA technology.

In one embodiment, a nucleic acid encoding an IFN-β therapeutic is constructed by isolating or synthesizing a DNA sequence encoding an IFN-β or variant thereof. For example, an IFN-β fusion protein can be produced as described, e.g., herein. A naturally-occurring IFN-β nucleic acid can be obtained according to methods well known in the art. For example, a nucleic acid can be isolated by reverse transcriptase-polymerase chain reaction (RT-PCR) using RNA obtained from a cell known to express IFN-β, e.g., a leukocyte, and primers based on the sequence of the IFN-β gene, e.g., SEQ ID NO: 1. Nucleic acids encoding IFN-β proteins can also be isolated by screening libraries, e.g., cDNA libraries made from cells expressing INF-β, with a probe, e.g., an oligonucleotide comprising a portion of an IFN-β sequence.

Alternatively, the complete amino acid sequence may be used to construct a back-translated gene. A DNA oligomer containing a nucleotide sequence coding for IFN-β therapeutic may be synthesized. For example, several small oligonucleotides coding for portions of the desired polypeptide may be synthesized and then ligated together. The individual oligonucleotides typically contain 5′ or 3′ overhangs for complementary assembly.

Changes can be introduced into nucleic acids encoding IFN-β proteins by methods well known in the art. For example, changes can be made by site-specific mutagenesis, as described in, e.g., Mark et al., “Site-specific Mutagenesis Of The Human Fibroblast Interferon Gene”, Proc. Natl. Acad. Sci. USA, 81, pp. 5662-66 (1984) and U.S. Pat. No. 4,588,585.

Another method of constructing a nucleic acid encoding an IFN-β therapeutic is via chemical synthesis. For example, a gene that encodes the desired IFN-β therapeutic may be synthesized by chemical means using an oligonucleotide synthesizer. Such oligonucleotides are designed based on the amino acid sequence of the desired IFN-β therapeutic.

When choosing a nucleic acid for expression in an expression system, it may be desirable to select those codons that are favored in the host cell or expression system in which the recombinant IFN-β therapeutic will be produced. It is known, e.g., that certain codons are expressed preferably over others in prokaryotic cells (“codon preference”).

A DNA sequence encoding an IFN-β therapeutic may or may not also include a DNA sequence that encodes a signal sequence. Such signal sequence, if present, should be one recognized by the cell chosen for expression of the IFN-β therapeutic. The signal sequence may be prokaryotic, eukaryotic or a combination of the two. Signal sequences are well known in the art, and several different ones are described in the art The signal sequence may be that of a native (i.e., naturally-occurring) IFN-β. The inclusion of a signal sequence depends on whether it is desired to have the IFN-β therapeutic secreted from the recombinant cells in which it is produced. If the chosen cells are prokaryotic, it generally is preferred that the DNA sequence not encode a signal sequence. If the chosen cells are eukaryotic, it generally is preferred that a signal sequence be encoded and most preferably that the wild-type IFN-β signal sequence be used.

Once assembled (by synthesis, site directed mutagenesis or another method), the nucleic acid encoding an IFN-β therapeutic is inserted into an expression vector, in which it is operatively linked to an expression control sequence appropriate for expression of the IFN-β therapeutic in the desired transformed host. Proper assembly may be confirmed by nucleotide sequencing, restriction mapping, and expression of a biologically active polypeptide in a suitable host or host cell. As is well known in the art, in order to obtain high expression levels of a transfected gene in a host or host cell, the gene must be operatively linked to transcriptional and translational expression control sequences that are functional in the chosen expression host.

The choice of expression control sequence and expression vector will depend upon the choice of host cell. A wide variety of expression host/vector combinations may be employed. Useful expression vectors for eukaryotic hosts, e.g., eukaryotic host cells, include, for example, vectors comprising expression control sequences from SV40, bovine papilloma virus, adenovirus and cytomegalovirus, e.g., the following vectors: pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems, see Molecular Cloning A Laboratory Manual, 2^{nd} Ed., ed by Sambrook, Fritsch and Maniatis (Cold Spring Harbor Laboratory Press: 1989) Chapters 16 and 17.

Useful expression vectors for bacterial hosts include known bacterial plasmids, such as plasmids from E. coli, including col E1, pCR1, pBR322, pMB9 and their derivatives, wider host range plasmids, such as RP4, phage DNAs, e.g., the numerous derivatives of phage lambda, e.g., NM989, and other DNA phages, such as M13 and filamentous single stranded DNA phages. Useful expression vectors for yeast cells include the 2.mu. plasmid and derivatives thereof. Useful vectors for insect cells include pVL 941. See also, Cate et al., “Isolation Of The Bovine And Human Genes For Mullerian Inhibiting Substance And Expression Of The Human Gene In Animal Cells”, Cell, 45, pp. 685-98 (1986).

In addition, any of a wide variety of expression control sequences may be used in these vectors. Such useful expression control sequences include the expression control sequences associated with structural genes of the foregoing expression vectors. Examples of useful expression control sequences include, for example, the early and late promoters of SV40 or adenovirus, the lac system, the trp system, the TAC or TRC system, the major operator and promoter regions of phage lambda, for example PL, the control regions of fd coat protein, the promoter for 3-phosphoglycerate kinase or other glycolytic enzymes, the promoters of acid phosphatase, e.g., Pho5, the promoters of the yeast α-mating system and other sequences known to control the expression of genes of prokaryotic or eukaryotic cells or their viruses, and various combinations thereof.

Any suitable host may be used to produce IFN-β therapeutics, including bacteria, fungi (including yeasts), plant, insect, mammal, or other appropriate animal cells or cell lines, as well as transgenic animals or plants. Exemplary hosts include strains of E. coli, Pseudomonas, Bacillus, Streptomyces, fungi, yeast, insect cells such as Spodoptera fruaiperda (SF9), animal cells such as Chinese hamster ovary (CHO) and mouse cells such as NS/0, African green monkey cells such as COS 1, COS 7, BSC 1, BSC 40, and BMT 10, and human cells, as well as plant cells in tissue culture. Such cells can be obtained from the American Type Culture Collection (ATCC). Preferred host cells for animal cell expression include cultured CHO cells and COS 7 cells and particularly the CHO-DDUKY-β1 cell line.

It should of course be understood that not all vectors and expression control sequences will function equally well to express the DNA sequences described herein. Neither will all hosts function equally well with the same expression system. However, one of skill in the art may make a selection among these vectors, expression control sequences and hosts without undue experimentation. The vector's copy number, the ability to control that copy number, and the expression of any other proteins encoded by the vector, such as antibiotic markers, should also be considered. For example, preferred vectors for use in this invention include those that allow the DNA encoding the IFN-β therapeutic to be amplified in copy number. Such amplifiable vectors are well known in the art. They include, for example, vectors able to be amplified by DHFR amplification (see, e.g., Kaufman, U.S. Pat. No. 4,470,461, Kaufman and Sharp, “Construction Of A Modular Dihydrafolate Reductase cDNA Gene: Analysis Of Signals Utilized For Efficient Expression”, Mol. Cell. Biol., 2, pp. 1304-19 (1982)) or glutamine synthetase (“GS”) amplification (see, e.g., U.S. Pat. No. 5,122,464 and European published application 338,841).

In selecting an expression control sequence, a variety of factors should also be considered. These include, for example, the relative strength of the sequence, its controllability, and its compatibility with the actual DNA sequence encoding the IFN-β therapeutic, particularly as regards potential secondary structures. Hosts should be selected by consideration of their compatibility with the chosen vector, the toxicity of the product coded for by the DNA sequences of this invention, their secretion characteristics, their ability to fold the polypeptides correctly, their fermentation or culture requirements, and the ease of purification of the products coded for by the DNA sequences.

Within these parameters, one of skill in the art may select various vector/expression control sequence/host combinations that will express the desired DNA sequences on fermentation or in large scale animal culture, for example, using CHO cells or COS 7 cells. Use of the CHO cell line CHO-KUKX-B1 DHFR sup for expressing INF-β variants is further described in U.S. Pat. No. 6,127,332.

An IFN-β therapeutic can also be produced in an in vitro system, e.g., in a in vitro translation system, e.g., cell lysate, e.g., a reticulocyte lysate. The term “in vitro translation system”, which is used herein interchangeably with the term “cell-free translation system” refers to a translation system which is a cell-free extract containing at least the minimum elements necessary for translation of an RNA molecule into a protein. In vitro translation systems typically comprise macromolecules, such as enzymes, translation, initiation and elongation factors, chemical reagents, and ribosomes. For example, an in vitro translation system may comprise at least ribosomes, ^{t}RNAs, initiator methionyl-^{t}RNA^{Met}, proteins or complexes involved in translation, e.g., eIF₂, eIF₃, the cap-binding (CB) complex, comprising the cap-binding protein (CBP) and eukaryotic initiation factor 4F (eIF_{4F}). A variety of in vitro translation systems are well known in the art and include commercially available kits. Examples of in vitro translation systems include eukaryotic lysates, such as rabbit reticulocyte lysates, rabbit oocyte lysates, human cell lysates, insect cell lysates and wheat germ extracts. Lysates are commercially available from manufacturers such as Promega Corp., Madison, Wis.; Stratagene, La Jolla, Calif; Amersham, Arlington Heights, Ill.; and GIBCO/BRL, Grand Island, N.Y. RNA for use in in vitro translation systems can be produced in vitro, e.g., using SP6 or T7 promoters, according to methods known in the art.

In another method, an IFN-β therapeutic is expressed from the endogenous gene in a host cell. The method may comprise inserting a heterologous promoter upstream of the coding region of the IFN-β gene, e.g., an inducible promoter, expressing the endogenous IFN-β gene and recovering the IFN-β produced. A heterologous promoter can be introduced into cells by “knock-in,” according to methods known in the art, or alternatively, by insertion of the promoter within the IFN-β gene.

The IFN-β therapeutic obtained according to the present invention may be glycosylated or unglycosylated depending on the host organism used to produce the therapeutic. If bacteria are chosen as the host, then the IFN-β therapeutic produced will be unglycosylated. Eukaryotic cells, on the other hand, will glycosylate the IFN-β therapeutics.

The IFN-β therapeutic produced by the transformed host can be purified according to any suitable method. Various methods are known for purifying IFN-β. See, e.g., U.S. Pat. Nos. 4,289,689, 4,359,389, 4,172,071, 4,551,271, 5,244,655, 4,485,017, 4,257,938, 4,541,952 and 6,127,332. In a preferred embodiment, the IFN-β therapeutic is purified by immunoaffinity, as described, e.g., in Okamura et al., “Human Fibroblastoid Interferon: Immunosorbent Column Chromatography And N-Terminal Amino Acid Sequence.” Biochem., 19, pp. 3831-35 (1980).

For example, the IFN-β proteins and variants thereof may be isolated and purified in accordance with conventional conditions, such as extraction, precipitation, chromatography, affinity chromatography, electrophoresis or the like. For example, the interferon proteins and fragments may be purified by passing a solution thereof through a column having an interferon receptor immobilized thereon (see U.S. Pat. No. 4,725,669). The bound interferon molecule may then be eluted by treatment with a chaotropic salt or by elution with aqueous acetic acid. The immunoglobulin fusion proteins may be purified by passing a solution containing the fusion protein through a column which contains immobilized protein A or protein G which selectively binds the Fc portion of the fusion protein. See, for example, Reis, K. J., et al., J. Immunol. 132:3098-3102 (1984); PCT Application, Publication No. W087/00329. The chimeric antibody may then be eluted by treatment with a chaotropic salt or by elution with aqueous acetic acid.

Alternatively the interferon proteins and immunoglobulin-fusion molecules may be purified on anti-interferon antibody columns, or on anti-immunoglobulin antibody columns to give a substantially pure protein. By the term “substantially pure” is intended that the protein is free of the impurities that are naturally associated therewith. Substantial purity may be evidenced by a single band by electrophoresis.

IFN-β that has been produced and purified can be characterized, e.g., by peptide mapping. For example, an IFN-β therapeutic sample can be digested with endoproteinase Lys-C and analyzed on a reverse phase HPLC, as described, e.g., in U.S. Pat. No. 6,127,332.

In a preferred embodiment, the IFN-β therapeutic is substantially free of other cellular material, e.g., proteins. The terms “purified preparations of an IFN-β therapeutic” refers to preparations of an IFN-β therapeutic having less than about 20% (by dry weight) contaminating cellular material, e.g., nucleic acids, proteins, and lipids, and preferably having less than about 5% contaminating cellular material. Preferred preparations of the IFN-β therapeutic have less than about 2% contaminating cellular material; even more preferably less than about 1% contaminating cellular material and most preferably less than about 0.5; 0.2; 0.1; 0.01; 0.001% contaminating cellular material.

Preferred IFN-β therapeutic compositions are also substantially free of other cellular proteins (also referred to herein as “contaminating proteins”), i.e., the compositions have less than about 20% (by dry weight) contaminating protein, and preferably having less than about 5% contaminating protein. Preferred preparations of the subject polypeptides have less than about 2% contaminating protein; even more preferably less than about 1% contaminating protein and most preferably less than about 0.5; 0.2; 0.1; 0.01; 0.001% contaminating proteins.

The purity and concentration of IFN-β preparations can be determined according to methods known in the art, e.g., by subjecting samples to gel electrophoresis, and as described, e.g., in Robert K. Scopes, Protein Purification, Principles and Practice, Third Ed., Springer Verlag New York, 1993, and references cited therein.

The biological activity of IFN-β therapeutics can be assayed by any suitable method known in the art, e.g., antibody neutralization of antiviral activity, induction of protein kinase, oligoadenylate 2,5-A synthetase or phosphodiesterase activities, e.g., as described in EP-B1-41313 and WO 00/23472. Such assays also include immunomodulatory assays (see, e.g., U.S. Pat. No. 4,753,795), growth inhibition assays, and measurement of binding to cells that express interferon receptors. Exemplary antiviral assays are further described in U.S. Pat. No. 6,127,332 and WO 00/23472.

The ability of IFN-β therapeutics to treat glomerulonephritis can also be assessed in animal models, e.g., those described in the Examples and further herein. The testing can be conducted, e.g., as described in the Examples.

IFN-β therapeutics can also be purchased commercially under the following brand names: AVONEX® (INF-β-1a) (Biogen, Inc., Cambridge, Mass.); REBIF® (IFN-β-1a) (Serono, S. A., Geneva, Switzerland); BETAFERON® or Bferon (IFN-β-1b) (Schering Aktiengesellschaft, Berlin, Germany); and BETASERON® or Bseron (Berlex, Montville, N.J.; IFN-β-1b). AVONEX® and REBIF® are recombinant human glycosylated IFN-β produced in Chinese hamster ovary cells. BETAFERON® and BETASERON® are produced in bacteria.

4. Methods of Treatment With IFN-β Therapeutics

The invention provides methods for treating glomerulonephritis or chronic renal failure in a subject having or likely to develop glomerulonephritis, comprising administering to the subject a therapeutically effective amount of an IFN-β therapeutic. The subject may be a subject who has been identified as having glomerulonephritis or chronic renal failure.

Glomerulonephritis, also referred to as “acute nephritis” or “acute glomerulonephritis” is an acute, but transient inflammatory process that affects the glomeruli, resulting in acute reductions of GFR, a resultant fluid imbalance and electrolyte abnormality. Symptoms of glomerulonephritis include: proteinuria; reduced glomerular filtration rate (GFR); serum electrolyte changes including azotemia (uremia, excessive blood urea nitrogen—BUN) and salt retention, leading to water retention resulting in hypertension and edema; hematuria and abnormal urinary sediments including red cell casts; hypoalbuminemia; hyperlipidemia; and lipiduria.

A number of diseases, e.g., set forth below, involve glomerulonephritis. If sufficiently severe, acute glomerulonephritis may result in acute or rapidly progressive renal failure. Acute glomerulonephritis associated with rapidly progressive renal failure is a common scenario that may be termed rapidly progressive glomerulonephritis because of its clinical behavior. Since damage to the glomerular wall is a consistent finding in acute glomerulonephritis, red cells and albumin will enter Bowman's space and pass into the urine. The combination of red cells in the urine, renal failure and fluid homeostatic abnormalities is called the nephritic syndrome. Massive loss of plasma proteins may result in a condition called the nephrotic syndrome, where the proteins lost in the urine deplete the serum protein balance, leading to low serum albumin, lipid abnormalities and edema. Laboratory findings of proteinuria (albuminuria) and hematuria, generally with red blood cell casts, are therefore necessary for a diagnosis of acute glomerulonephritis, while the absence of these findings suggest other diagnoses. For example, tubulointerstitial nephritis involves a transient acute inflammation of the renal tubules and interstitium without involving the glomerular capillaries. As in acute glomerulonephritis, hematuria, red blood cell casts and reduction of GFR occur, but proteinuria is less marked, involving mainly low molecular weight proteins instead of albumin.

A number of disease entities may be responsible for the syndrome of acute glomerulonephritis. Renal biopsy is usually required to evaluate patients with acute glomerulonephritis, whether or not a degree of renal failure is present. Diagnosis, prognosis and therapy are all determined by the precise histologic and ultrastructural patterns identified on renal biopsy. Furthermore, biopsy tissues may be analyzed to determine the types of immune complexes, immunoglobulins and other substances involved in a particular glomerulonephritis, with immunofluorescence analysis commonly being performed. Diseases affecting the kidney may be categorized according to their pathogenesis, whether or not they result in sufficient nephron damage to affect the glomerular filtration rate and thus cause some type of renal failure.

A traditional nomenclature has arisen to describe various features of glomerular disease. Glomerular disease, glomerulopathy, and glomerulonephritis may be used interchangeably in the literature, although the term glomerulonephritis frequently connotes an inflammatory process, as discussed above. Glomerular diseases are classified as primary when the pathology arises in the kidney and leads therefrom to systemic manifestations; glomerular diseases are termed secondary when they result from some other, multisystem disorder. Pathological features seen on light microscopy allow further characterization of the type of glomerular disease. A lesion affecting part of the glomerular tuft is termed segmental, while a lesion affecting almost all of the glomerular tuft is called global. Abnormalities characterized by an increase in the number of cells in a glomerulus are termed proliferative, whether the increase in cell number is due to infiltration of leukocytes or proliferation of resident glomerular cells. Cell proliferation involving the Bowman's capsule cells is called extracapillary, proliferation involving the endothelial or mesangial cells is termed the intracapillary or endocapillary. A collection of cells collecting in Bowman's space and formed in a half-moon shape is called a crescent, and usually is composed of proliferating parietal epithelial cells and infiltrating monocytes. Crescentic glomerulonephritis is a type of acute glomerulonephritis characterized by crescent formation in the glomeruli. Since this condition is often associated with rapidly progressive renal failure, the term crescentic glomerulonephritis may be used interchangeably with rapidly progressive glomerulonephritis. If a glomerular disease is characterized by the expansion of the glomerular basement membrane by immune deposits, it is termed membranous. Combinations of the aforesaid terms are used to describe glomerular disease entities based on their dominant pathological features. Proliferative glomerulopathies, also called inflammatory glomerulopathies, include such conditions as focal proliferative glomerulonephritis, diffuse proliferative glomerulonephritis, mesangial proliferative glomerulonephritis, and crescentic glomerulonephritis, each term suggesting the location and/or type of proliferating cell. These conditions are characterized by blood cells and proteins in the urine sediment, but without the amount of proteins loss that would cause the nephrotic syndrome, a so-called “nephritic” picture. Membranous glomerulopathies involves a change to the glomerular filtration barrier for proteins, including the glomerular basement membrane and the visceral epithelial cells. These disorders, including membranous glomerulopathy, minimal change disease, and focal and segmental glomerulosclerosis, lead to heavy protein loss that may result in nephrotic syndrome. As the name suggests, membranoproliferative glomerulonephritis is a hybrid disorder with clinical features suggesting both cellular proliferation and altered glomerular filtration barrier. Those disorders characterized by prominent extravascular deposition of proteinaceous or fibrillar material are called glomerular deposition diseases. They may include both nephritic and nephrotic components, thus overlapping with the findings in proliferative or membranous disorders. A final category of diseases affecting the kidney are the thrombotic microangiopathies, disorders in which clotting takes place within the renal microvasculature. Each of these categories has a particular type of etiology.

A spectrum of proliferative glomerulopathies exists, suggesting that different histopathologic features result from different inflammatory processes. For example, diffuse proliferative glomerulonephritis may represent an acute immune response to a sudden heavy antigen load. Crescentic glomerulonephritis may involve a less dramatic immune response to a smaller antigen challenge in individuals who have been presensitized. Focal proliferative or mesangial proliferative glomerulonephritis represents the least aggressive end of the spectrum, where patients may experience only slowly progressive renal insufficiency.

Immunofluorescence studies of renal biopsies help distinguish the major causes of proliferative glomerulopathy. There are three broad diagnostic categories, each associated with a particular pattern of immunoglobulin deposition visible on immunofluorescence combined with a vigorous cellular proliferation. Granular deposits of immunoglobulin characterize the first category: immune-complex glomerulonephritis. Linear deposition of immunoglobulin along the glomerular basement membrane characterize the second category: anti-GBM disease. Minimal immunoglobulin deposition characterizes the third category: pauci-immune glomerulonephritis. The immune-complex glomerulonephritis may represent a response to a known antigenic stimulus (e.g. poststreptococcal glomerulonephritis), or may form part of a multisystem immune-complex disorder (e.g., lupus, cryoglobulinemia, or bacterial endocarditis); in certain cases, no cause can be determined and the disease is considered idiopathic. Anti-GBM disease is a rare disorder in which autoantibodies are formed that attack the Type IV collagen. The majority of patients with anti-GBM disease also have lung hemorrhage, a condition called Goodpasture's syndrome. Pauci-immune glomerulonephritis is characterized by abnormal levels of circulating antineutrophil cytoplasmic antibody, implying some dysregulation of humoral immunity.

Immunologically mediated glomerulonephritis accounts for a large fraction of acquired renal disease. Generally there is a deposition of antibodies in the glomerular tuft, often autoantibodies. Cellular immune mechanisms involved in antibody-mediated glomerulonephritis further modulate antibody production and induce antibody-dependent cytotoxicity. Most antibody-mediated glomerulonephritis in patients is initiated by the reaction of circulating antibodies with autoantigens.

Antibodies may be found in the glomerulus as a result of several different processes. First, circulating autoantibodies may react with intrinsic autoantigens that are components of the normal glomerulus. Second, circulating autoantibodies and extrinsic antigens that have been deposited within the glomerulus may lead to the in situ formation of glomerular immune complexes. Third, immune complexes formed in the systemic circulation may be trapped within the glomerulus. The location for antibody deposition will determine to a great extent the clinical features of the glomerular disease. Acute deposition of antibody in the subendothelial space or mesangium can trigger a vigorous nephritic response characterized by rapid recruitment of leukocytes and platelets from the glomerular capillaries. Antibody deposition in the subepithelial space typically induces a nephrotic type response characterized by proteinuria with less vigorous inflammatory cell infiltrate.

Any of these immunologic processes may set off a cascade of inflammatory reactions within the glomerulus, resulting in glomerular injury and subsequent repair. The reactivity of autoantibodies to intrinsic or planted glomerular antigens leads to the production of complement, chemoattractants, chemokines and cytokines. Complement dependent and complement independent mechanisms are thereby initiated, resulting in damage to the glomerular cells. Leukocytes and platelets are also recruited to the glomerulus, triggering further injury. Sustained immune complex deposition over months to years can also provoke a marked increase in basement membrane production. The resolution process for any immune-mediated glomerulopathy cannot take place until local immune activity ceases, with no further antibody production or immune complex formation, with removal of deposited and circulating immune complexes, with prevention of further inflammatory cell recruitment, with dissipation of inflammatory mediators in the renal tissues, and with normalization of vascular tone and endothelial adhesiveness.

Following the glomerular injury, there may be healing with scarring. Recovery may be complete, without residual impairment. More commonly, glomerular scarring is widespread, with an impact on renal function. It has been recognized that transforming growth factor β (TGF-β), a cytokine that accompanies the healing process in the glomerulus, stimulates production of extracellular matrix and inhibits synthesis of tissue proteases that degrade matrix proteins, thereby enhancing scar formation after glomerular injury. Scarring following glomerular injury further damages the residual viable nephrons, leading to progressing nephron loss. As more functioning nephrons are lost, the remaining nephrons compensate, as described above, a process that damages them as well. The end result may be a progressive decrease in renal function, culminating in chronic renal failure with its final stage of end-stage renal disease.

IFN-β therapeutics can also be used to treat focal glomeruloscerosis and collapsing glomerulopathies, including the idiopathic and secondary forms due to HIV infection. Collapsing glomerulonephritis is a rapidly progressive disease leading to renal failure that has no effective therapy. This disease occurs mostly in HIV patients. Since proteinurea plays a major role in these diseases, it is expected that IFN-β therapeutics, which significantly reduce proteinurea, will have a significant effect on improving these diseases. Another disease in which proteinurea plays a major role and in which IFN-β therapeutics are expected to be useful is minimal change disease, also referred to as minimal change nephropathy (MCN) and minimal change nephrotic syndrome (MCNS).

Accordingly IFN-β therapeutics can be used for treating renal conditions associated with inflammation of glomeruli, e.g., any of the following renal conditions: focal glomeruloscerosis and collapsing glomerulopathies, minimal change disease, acute glomerulonephritis, crescentic glomerulonephritis, nephritic syndrome, nephrotic syndrome, primary glomerulonephritis, secondary glomerulonephritis, proliferative glomerulonephritis, membraneous glomerulonephritis, membranoproliferative glomerulonephritis, immune-complex glomerulonephritis, anti-glomerular basement membrane (anti-GBM) glomerulonephritis, pauci-immune glomerulonephritis, diabetic glomerulopathy, chronic glomerulonephritis, and hereditary nephritis. Any disease or condition resulting from these renal diseases, such as chronic renal disease and end-stage renal disease, can also be treated according to the methods of the invention.

5. Subjects for Treatment

As a general matter, the methods of the present invention may be utilized for any mammalian subject having, or at risk of developing, glomerulonephritis, chronic renal failure, or at risk for renal replacement therapy (i.e., chronic dialysis or renal transplant). A “subject having, or at risk of developing, glomerulonephritis or chronic renal failure” is a subject that is reasonably expected to suffer a progressive loss of renal function associated with progressive loss of functioning nephron units. Whether a subject has or is at risk of developing glomerulonephritis or chronic renal failure is a determination that may routinely be made by one of ordinary skill in the relevant medical or veterinary art. Subjects having, or at risk of developing, glomerulonephritis or chronic renal failure (or at risk of the need for renal replacement therapy) include, but are not limited to, the following: subjects which may be regarded as afflicted with chronic renal, failure, end-stage renal disease, chronic diabetic nephropathy, hypertensive nephrosclerosis, chronic glomerulonephritis, hereditary nephritis, and/or renal dysplasia; subjects having proteinuria, serum electrolyte changes, e.g., azotemia (uremia, i.e., excessive blood urea nitrogen or “BUN”); salt retention, resulting in hypertension and edema, hematuria and abnormal urinary sediments including red cell casts; hypoalbuminemia, hyperlipidemia and lipiduria; subjects having a biopsy indicating glomerular hypertrophy, tubular hypertrophy, chronic glomerulosclerosis, and/or chronic tubulointerstitial sclerosis; subjects having an ultrasound, MRI, CAT scan, or other non-invasive examination indicating renal fibrosis or smaller than normal kidneys. Further indications of subjects having, or at risk of developing glomerulonephritis or CRF, are well known to workers having ordinary skill in the art. For example, all the following may be criteria to determine if a subject has, or is at risk of developing glomerulonephritis or CRF: subjects having an unusual number of broad casts present in urinary sediment; subjects having a GFR which is chronically less than about 50%, and more particularly less than about 40%, 30% or 20%, of the expected GFR for the subject; human male subjects weighing at least about 50 kg and having a GFR which is chronically less than about 50 ml/min, and more particularly less than about 40 ml/min, 30 ml/min or 20 ml/min; human female subjects weighing at least about 40 kg and having a GFR which is chronically less than about 40 ml/min, and more particularly less than about 30 ml/min, 20 ml/min or 10 ml/min; subjects possessing a number of functional nephron units which is less than about 50%, and more particularly less than about 40%, 30% or 20%, of the number of functional ephron units possessed by a healthy but otherwise similar subject, subjects which have a ingle kidney; and subjects which are kidney transplant recipients.

Mammalian subjects which may be treated include, but are not limited to, human subjects or patients. In addition, the invention may be employed in the treatment of domesticated mammals which are maintained as human companions (e.g., dogs, cats, horses), which have significant commercial value (e.g., dairy cows, beef cattle, sporting animals), which have significant scientific value (e.g., captive or free specimens of endangered species), or which otherwise have value. The subjects for treatment need not present indications for treatment with IFN-β therapeutic other than those indications associated with risk of glomerulonephritis, chronic renal failure or end-stage renal disease, e.g., in need of renal replacement therapy. That is, the subjects for treatment are expected to be otherwise free of indications for treatment with IFN-β therapeutics. In some cases, however, the subjects may present with other symptoms (e.g., viral disease, such as hepatitis infection) for which treatment with IFN-β therapeutics would be indicated. In such cases, the treatment should be adjusted accordingly so to avoid excessive dosing.

One of ordinary skill in the medical or veterinary arts is trained to recognize subjects which may be at a substantial risk of glomerulonephritis, chronic renal failure, or at substantial risk for renal replacement therapy. In particular, clinical and non-clinical trials, as well as accumulated experience, relating to the presently disclosed and other methods of treatment, are expected to inform the skilled practitioner in deciding whether a given subject has, or is at risk of developing, glomerulonephritis, chronic renal failure, or at risk of needing renal replacement therapy, and whether any particular treatment is best suited to the subjects needs, including treatment according to the present invention.

As a general matter, a mammalian subject may be regarded as having, or at risk of developing, glomerulonephritis, chronic renal failure, or at risk of needing renal replacement therapy, if that subject has already been diagnosed as afflicted with, or would be regarded as being afflicted with, a condition which typically leads to progressive loss of renal function associated with progressive loss of functioning nephron units. Such conditions include, but are not limited to, end-stage renal disease, chronic diabetic nephropathy, diabetic glomerulopathy, diabetic renal hypertrophy, hypertensive nephrosclerosis, hypertensive glomerulosclerosis, chronic glomerulonephritis, hereditary nephritis, renal dysplasia and chronic rejection following renal allograft transplantation and the like. These, and other diseases and conditions known in the art, typically lead to a progressive loss of functioning nephrons and to the onset of chronic renal failure.

Frequently, one of skill in the medical or veterinary arts may base a prognosis, diagnosis or treatment decision upon an examination of a renal biopsy sample. Such biopsies provide a wealth of information useful in diagnosing disorders of the kidney. Subjects having, or at risk of developing, glomerulonephritis, chronic renal failure, or at risk of needing renal replacement therapy, may be recognized by histological indications from renal biopsies including, but not limited to, the presence of inflammatory cells, e.g., T cells and macrophages, in the glomeruli, glomerular hypertrophy, tubular hypertrophy, glomerulosclerosis, tubulointerstitial sclerosis, and the like.

Less invasive techniques for assessing kidney morphology include MRI, CAT and ultrasound scans. Scanning techniques are also available which employ contrasting or imaging agents (e.g., radioactive dyes) but, it should be noted, some of these are particularly toxic to renal tissues and structures and, therefore, their use may be ill-advised in subjects having, or at risk of developing glomerulonephritis or chronic renal failure. Such non-invasive scanning techniques may be employed to detect conditions such as renal fibrosis or sclerosis, focal renal necrosis, renal cysts, and renal gross hypertrophy which will place a subject in the category of having or at risk of developing glomerulonephritis, chronic renal failure, or at risk of needing renal replacement therapy.

Frequently, prognosis, diagnosis and/or treatment decisions are based upon clinical indications of renal function. One such indication is the presence in urinary sediment of an unusual number of “broad” or “renal failure” casts, which is indicative of tubular hypertrophy and suggests the compensatory renal hypertrophy which typifies chronic renal failure. Another indication of renal function is the glomerular flow rate (GFR), which can be measured directly by quantifying the rate of clearance of particular markers, or which may be inferred from indirect measurements.

The methods of treatment of the present invention need not be restricted to subjects presenting with any particular measures of GFR, or any other particular marker of renal function. Indeed, it is not necessary that the GFR of a subject, or any other particular marker of renal function, be determined before practicing the treatments of the present invention. Nonetheless, the measurement of GFR is considered to be a preferred means of assessing renal function.

As is well known in the art, GFR reflects the rate of clearance of a reference or marker compound from the plasma to the urine. The marker compound to be considered is typically one which is freely filtered by the glomeruli, but which is not actively secreted or reabsorbed by the renal tubules, and which is not significantly bound by circulating proteins. The rate of clearance is typically defined by the formula, presented above, which relates the volume of urine produced in a twenty-four period, and the relative concentrations of the marker in the urine and plasma. To be more accurate, the GFR should also be corrected for body surface area. The “gold standard” reference compound is insulin because of its filtration properties and lack of serum-binding. The concentration of this compound is, however, difficult to quantify in blood or urine. The clearance rates of other compounds, including creatinine, are therefore often used instead of insulin. In addition, various formulas are often employed which seek to simplify the estimation of actual GFR by omitting considerations of actual urine concentrations of the marker, actual daily volumes of urine produced, or actual body surface area. These values may be replaced by estimates based on other factors, by baseline values established for the same subject, or by standard values for similar subjects. These estimates should be used with caution, however, as they may entail inappropriate assumptions based upon the renal function of normal or healthy subjects. In addition, clearance of p-aminohippurate (PAH) is used to estimate renal clearance rates.

Various methods and formulas have been developed in the art which describe an expected value of GFR for a healthy subject with certain characteristics. In particular, formulas are available which provide an expected value of the GFR based upon plasma creatinine levels, age, weight and sex (see, e.g., “definitions” section herein). Other formulas may, of course, be employed and tables of standard values may be produced for subjects of a given age, weight, sex, and/or plasma creatinine concentration. Newer methods of measuring or estimating GFR (e.g., using NMR or MRI technologies) are also now available in the art and may be used in accordance with the present invention (see, e.g., U.S. Pat. Nos. 5,100,646 and 5,335,660).

As a general matter, irrespective of the manner in which GFR is measured or estimated, a subject may be considered to have, or be at risk of developing, glomerulonephritis, chronic renal failure, or at risk of needing renal replacement therapy, when the subject has a GFR which is chronically less than about 50% of the expected GFR for that subject. The risk is considered greater as the GFR falls lower. Thus, a subject is increasingly considered at risk if the subject has a GFR which is chronically less than about 40%, 30% or 20% of the expected GFR. A human male subject weighing at least about 50 kg may be considered to be in, or at risk of, glomerulonephritis, chronic renal failure, or at risk of needing renal replacement therapy, when the subject has a GFR that is chronically less than about 50 ml/min. The risk is considered greater as the GFR falls lower. Thus, a subject is increasingly considered at risk if the subject has a GFR that is chronically less than about 40, 30 or 20 ml/min. A human female subject weighing at least about 40 kg may be considered to be in, or at risk of, glomerulonephritis, chronic renal failure, or at risk of needing renal replacement therapy, when the subject has a GFR that is chronically less than about 40 ml/min. The risk is considered greater as the GFR falls lower. Thus, a subject is increasingly considered at risk if the subject has a GFR that is chronically less than about 30, 20 or 10 ml/min. As a general matter, a subject may be regarded to be in, or at risk of, glomerulonephritis, chronic renal failure, or at risk of needing renal replacement therapy, if that subject possesses a number of functional nephron units which is less than about 50% of the number of functional nephron units of a healthy, but otherwise similar, subject. As above, the risk is considered greater as the number of functional nephrons decreases further. Thus, a subject is increasingly considered at risk if the subject has a number of functional nephrons which is less than about 40, 30 or 20% of the number for a similar but healthy subject.

Finally, it should be noted that subjects possessing a single kidney, irrespective of the manner of loss of the other kidney (e.g., physical trauma, surgical removal, birth defect), may be considered to be prima facie at risk of glomerulonephritis, chronic renal failure, or the need for renal replacement therapy. This is particularly true for those subjects in which one kidney has been lost due to a disease or condition which may afflict the remaining kidney. Similarly, subjects which are already recipients of a renal transplant, or which are already receiving chronic dialysis (e.g., chronic hemodialysis or continuous ambulatory peritoneal dialysis) may be considered to be at risk of glomerulonephritis, chronic renal failure, or the need for further renal replacement therapy.

Subjects that can be treated according to the methods of the invention also include those having a condition or disease that is known to be treatable with IFN-β, e.g., multiple sclerosis and viral infections. Exemplary viral infections include hepatitis, e.g., hepatitis B infections. In such situations, a regimen of IFN-β therapeutic administration may be developed that is adapted for treating both conditions. Subjects may also be subjects who do not have a viral infection that can be treated with IFN-β or a viral infection causing glomerulonephritis. Accordingly, exemplary subjects include those who do not harbor a hepatitis virus, e.g., hepatitis B or C virus, or wherein the glomerulonephritis was not caused by a hepatitis virus, e.g., hepatitis B or C virus. Alternatively, the subject may also be a subject having or likely to develop glomerulonephritis caused by a viral infection. In other embodiments, the subject does not have end-stage renal failure or renal cell carcinoma.

6. Formulations and Methods of Treatment

INF-β therapeutics may be administered by any route that is compatible with the particular renal therapeutic agent employed. Thus, as appropriate, administration may be oral or parenteral, including intravenous, intraperitoneal, and renal intracapsular routes of administration. In addition, administration may be by periodic injections of a bolus of the agent(s) described herein (i.e., IFN-β therapeutics), or may be made more continuous by intravenous or intraperitoneal administration from a reservoir which is external (e.g., an i.v. bag) or internal (e.g., a bioerodable implant or implanted pump). In a method according to the invention, INF-β therapeutics are preferably administered parenterally. The term “parenteral” as used herein includes aerosol, subcutaneous, intravenous, intramuscular, intra-articular, intrasynovial, intrasternal, intrathecal, intrahepatic, intralesional and intracranial injection or infusion techniques.

The agents of the invention may be provided to an individual by any suitable means, preferably directly (e.g., locally, as by injection or topical administration to a tissue locus) or systemically (e.g., parenterally or orally). Where the agent is to be provided parenterally, such as by intravenous, subcutaneous, or intramuscular, administration, the agent preferably comprises part of an aqueous solution. The solution is physiologically acceptable so that in addition to delivery of the desired agent to the subject, the solution does not otherwise adversely affect the subject's electrolyte and/or volume balance. The aqueous medium for the agent thus may comprise normal physiologic saline (e.g., 0.9% NaCl, 0.15M, pH 7-7.4).

The INF-β therapeutics are preferably administered as a sterile pharmaceutical composition containing a pharmaceutically acceptable carrier, which may be any of the numerous well known carriers, such as water, saline, phosphate buffered saline, dextrose, glycerol, ethanol, and the like, or combinations thereof. INF-β therapeutics may be prepared in a composition comprising one or more other proteins, e.g., for stabilizing the IFN-β therapeutic. For example, IFN-β therapeutics can be mixed with albumin.

Pharmaceutical compositions may comprise an IFN-β therapeutic together with any pharmaceutically acceptable carrier. The term “carrier” as used herein includes acceptable adjuvants and vehicles. Pharmaceutically acceptable carriers that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The IFN-β or variants thereof can also be administered in the form of liposome delivery systems, such as small unilamellar vesicles, large unilamellar vesicles and multilamellar vesicles. Liposomes can be formed from a variety of phospholipids, containing cholesterol, stearylamine, or phosphatidylcholines. In some embodiments, a film of lipid components is hydrated with an aqueous solution of drug to a form lipid layer encapsulating the drug, as described in U.S. Pat. No. 5,262,564.

IFN-βs or variants thereof may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. The IFN-βs or variants thereof can also be coupled to proteins, such as, for example, receptor proteins and albumin. Furthermore, the IFN-βs or variants thereof may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

According to this invention, the pharmaceutical compositions may be in the form of a sterile injectable preparation, for example a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as do natural pharmaceutically-acceptable oils, such as olive oil or castor oil, especially in their polyoxyethylated versions. These oil solutions or suspensions may also contain a long-chain alcohol diluent or dispersant.

Pharmaceutical compositions comprising INF-β therapeutics may also be given orally. For example, they can be administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added. Topically-transdermal patches may also be used.

In a preferred embodiment, an IFN-β or variant thereof is provided as a liquid composition comprising a stabilizing agent. The stabilizing agent may be present at an amount of between 0.3% and 5% by weight of the IFN-β or variant thereof. The stabilizing agent may be an amino acid, such as an acidic amino acid (e.g., glutamic acid and aspartic acid) or arginine or glycine. If the stabilizing agent is arginine-HCl, its concentration will preferably range between 0.5% (w/v) to 5% and is most preferably 3.13% (equivalent to 150 mM arginine-HCl). IF the stabilizing agent is glycine, its concentration will preferably range between 0.5% (w/v) to 2.0% and most preferably 0.52% (equivalent to 66.7 mM to 266.4 mM, and most preferably 70 mM). If the stabilizing agent is glutamic acid, its concentration will preferably range between 100 mM to 200 mM, and is most preferably 170 mM (equivalent to a w/v percent ranging from 1.47% to 2.94% and most preferably 2.5%). The preferred range of concentrations of IFN-β or variant thereof in the liquid formulations is from about 30 μg/ml to about 250 μg/ml. A preferred concentration range is 48 to 78 μg/ml and the most preferred concentration is about 60 μm/ml. In terms of International Standard values, the Biogen internal standard has been standardized to the WHO International Standard for Interferon, Natural #Gb-23-902-531, so that the range of concentration in IU (for a 0.5 ml injection volume) is from about 6 IMU to 50 IMU and the most preferred concentration is 12 IMU.

Preferably, the amino acid stabilizing agent is arginine which is incorporated as its acidic form (arginine-HCl) in about pH 5.0 solutions. Accordingly, poly-ionic excipients are preferred. Preferably the liquid composition is contained within a vessel, e.g., a syringe, in which the vessel has a surface in contact with the liquid that is coated with a material inert to IFN-β, e.g., silicone or polyetrafluoroethylene. Even more preferred compositions have a pH between 4.0 and 7.2. The solution comprising the stabilizing agent has preferably not been lyophilized and has not been subject to oxygen containing gas during preparation and storage.

The organic acid and phosphate buffers to be used in the present invention to maintain the pH in the range of about 4.0 to about 7.2, and preferably from about 4.5 to about 5.5, and most preferably 5,0, can be conventional buffers of organic acids and salts thereof such as citrate buffers (e.g., monosodium citrate-disodium citrate mixture, citric acid-trisodium citrate mixture, citric acid-monosodium citrate mixtures, etc.), succinate buffers (e.g., succinic acid-monosodium succinate mixture, succinic acid-sodium hydroxide mixture, succinic acid-disodium succinate mixture, etc.), tartrate buffers, fumarate buffers, gluconate buffers, oxalate buffers, lactate buffers, phosphate buffers, and acetate buffers, as further described in WO 98/28007.

Exemplary formulations, which can be prepared as described in WO 98/38007, include:

(i) a 20 mM acetate buffer at pH 5.0, the buffer having preferably not previously been lyophilized, in which the buffer includes IFN-β and at least one ingredient selected from (a) 150 mM arginine-HCl; (b) 100 mM sodium chloride and 70 mM glycine; (c) 150 mM arginine-HCl and 15 mg/ml human serum albumin; (d) 150 mM arginine-HCl and 0.1% Pluronic F-68; (e) 140 mM sodium chloride; (f) 140 mM sodium chloride and 15 mg/ml human serum albumin; and (g) 140 mM sodium chloride and 0.1% Pluronic F-68;

(ii) a liquid a pH 5.0 that includes IFN-β or a variant thereof, 170 mM L-glutamic acid, and 150 mM sodium hydroxide, the liquid preferably not having previously been lyophilized; and

(iii) a 20 mM phosphate buffer at pH 7.2, the buffer having preferably not previously been lyophylized, wherein the buffer includes IFN-β and least one ingredient selected from: (a) 140 mM arginine-HCl and (b) 100 mM sodium chloride and 70 mM glycine.

Prefered compositions also include polysorbate, e.g., at 0.005% w/v polysorbate 20.

IFN-βs can be formulated in dry powder form, which may or may not be solubilized or suspended prior to administration to a subject. In particular, it has been shown that IFN-βs conjugated to a polymer, e.g., PEG are particularly stable in dry form (see, e.g., WO 00/23114 and PCT/US/95/06008).

The pharmaceutical compositions of this invention may also be administered by nasal aerosol or inhalation through the use of a nebulizer, a dry powder inhaler or a metered dose inhaler. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. According to another embodiment compositions containing a compound of this invention may also comprise an additional agent selected from the group consisting of corticosteroids, anti-inflammatories, immunosuppressants, anti-metabolites, and immunomodulators. Compounds within each of these classes may be selected from any of those listed under the appropriate group headings in “Comprehensive Medicinal Chemistry”, Pergamon Press, Oxford, England, pp. 970-986 (1990), the disclosure of which is herein incorporated by reference. Specific compounds are theophylline, sulfasalazine and aminosalicylates (anti-inflammatories); cyclosporin, FK-506, and rapamycin (immunosuppressants); cyclophosphamide and methotrexate (antimetabolites); steroids (inhaled, oral or topical) and other interferons (immunomodulators).

Useful solutions for parenteral administration may be prepared by any of the methods well known in the pharmaceutical art, described, for example, in Remington's Pharmaceutical Sciences (Gennaro, A., ed.), Mack Pub., 1990.

Parental injectable administration is generally used for subcutaneous, intramuscular or intravenous injections and infusions. For example, when a subcutaneous injection is used to deliver 0.01-100 μg/kg, or more preferably 0.01-10 μg/kg of IFN-β, e.g., PEGylated IFN-β, over one week, two injections of 0.005-50 μg/kg, or more preferably 0.005-5 μg/kg, respectively, may be administered at 0 and 72 hours. Additionally, one approach for parenteral administration employs the implantation of a slow-release or sustained-released system, which assures that a constant level of dosage is maintained, according to U.S. Pat. No. 3,710,795, incorporated herein by reference.

As will be appreciated by one of ordinary skill in the art, the formulated compositions contain therapeutically effective amounts of the IFN-β therapeutic. That is, they contain amounts that provide appropriate concentrations of the IFN-β therapeutic to the renal tissues or other appropriate tissues for a time sufficient to prevent, inhibit, delay or alleviate permanent or progressive loss of renal function, or otherwise provide therapeutic efficacy. As will be appreciated by those skilled in the art, the concentration of the compounds described in a therapeutic composition of the present invention will vary depending upon a number of factors, including the biological efficacy of the selected agent, the chemical characteristics (e.g., hydrophobicity) of the compounds employed, the formulation of the compound excipients, the administration route, and the treatment envisioned, including whether the active ingredient will be administered directly into a kidney or renal capsule, or whether it will be administered systemically. The preferred dosage to be administered also is likely to depend on such variables such as the condition of the renal tissues, extent of renal function loss, and the overall health status of the particular subject. Dosages may be administered continuously, or daily, but it, is currently preferred that dosages be administered once, twice or three times per week for as long as a satisfactory response persists (as measured, for example, by stabilization and/or improvement of renal function by appropriate medical markers and/or quality of life indices). Less frequent dosages, for example monthly dosages, may also be employed. For subjects who would otherwise require continuous, bi-weekly or triweekly hemodialysis sessions, continuous, bi-weekly or triweekly intravenous or intraperitoneal infusions are not considered unduly inconvenient. In addition, in order to facilitate frequent infusions, implantation of a semi-permanent stent (e.g., intravenous, intraperitoneal or intracapsular) may be advisable.

The dosage regimen utilizing the IFN-β is selected in accordance with a variety of factors including type, species, age, weight, sex and medical condition of the patient; the severity of the condition to be treated; the route of administration; the renal and hepatic function of the patient; and the particular compound or salt thereof employed. The activity of the compounds of the invention and sensitivity of the patient to side effects are also considered. An ordinarily skilled physician or veterinarian can readily determine and prescribe the effective amount of the drug required to prevent, counter or arrest the progress of the condition.

Oral dosages of the present invention, preferably for pegylated INF-β therapeutics, will range between about 0.01-100 μg/kg/day orally, or more preferably 0.01-10 μg/kg/day orally. The compositions are preferably provided in the form of scored tablets containing 0.5-5000 μg, or more preferably 0.5-500 μg of active ingredient.

For any route of administration, divided or single doses may be used. For example, compounds of the present invention may be administered daily or weekly, in a single dose, or the total dosage may be administered in divided doses of two, three or four.

Any of the above pharmaceutical compositions may contain 0.1-99%, 1-70%, or, preferably, 1-50% of the active compounds of the invention as active ingredients.

The course of the disease and its response to drug treatments may be followed by clinical examination and laboratory findings. The effectiveness of the therapy of the invention is determined by the extent to which the previously described signs and symptoms of a condition, e.g., chronic hepatitis, are alleviated and the extent to which the normal side effects of interferon (i.e., flu-like symptoms such as fever, headache, chills, myalgia, fatigue, etc. and central nervous system related symptoms such as depression, paresthesia, impaired concentration, etc.) are eliminated or substantially reduced.

The IFN-β therapeutics may be administered alone or in combination with other molecules known to be beneficial in the treatment of the conditions described herein, e.g., anti-inflammatory drugs. When used in combination with other agents, it may be necessary to alter the dosages of the IFN-β therapeutics accordingly.

The amount of active ingredient that may be combined with the carrier materials to produce a single dosage form will vary depending upon the host treated, and the particular mode of administration. It should be understood, however, that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of active ingredient may also depend upon the therapeutic or prophylactic agent, if any, with which the ingredient is co-administered.

The effective dosage and dose rate of the IFN-β therapeutics will depend on a variety of factors, such as the nature of the inhibitor, the size of the patient, the goal of the treatment, the nature of the pathology to be treated, the specific pharmaceutical composition used, and the judgment of the treating physician. Dosage levels of between about 0.001 and about 100 mg/kg body weight per day, preferably between about 0.1 and about 50 mg/kg body weight of the active ingredient compound are useful. Most preferably, an IFN-β therapeutic will be administered at a dose ranging between about 0.1 mg/kg body weight and about 20 mg/kg body weight, preferably ranging between about 1 mg/kg body weight and about 3 mg/kg body weight and at intervals of every 1-14 days. Preferred dosages consist of an injection of about 6 MIU per week or three times per week. Optimization of dosages can be determined, e.g., by administration of the IFN-β therapeutics, followed by assessment of the circulating or local concentration of the IFN-β therapeutic.

In a most preferred embodiment, AVONEX® is administrated to subjects in need thereof. AVONEX® is sold as a lyophilized powder consisting of the following:
Formulation per 1 ml dose:
30 mcg interferon-b-1a (6 million international units (MIU))
50 mM sodium phosphate
100 mM, sodium chloride
15 mg Human Serum Albumin
pH 7.2
   The specific activity of AVONEX® interferon is 2×10⁸ units/mg, i.e., 200 MU of antiviral activity per milligram of IFN-b-1a protein. The patient reconstitues the powder with sterile water prior to intramuscular injection of the 1 ml once per week AVONEX® can also be prepared as a liquid formulation consisting of the following:
Formulation per 0.5 ml dose:
30 mcg (μg) IFN-b-1a (6 million international units (MIU))
20 mM acetate (sodium acetate and acetic acid)
150 mM arginine HCl
0.005% w.v polysorbate 20
water for injection
pH 4.8
   This formulation can be packaged in a pre-filled syringe. The patient may either manually use the syringe as provided or use in conjunction with an autoinjector. The dosing schedule is 6 MUI (i.e., 30 mcg) intramuscular once per week.

In another embodiment, the IFN-β is Rebif, which is provided as a lyophilized powder and as a liquid formulation. The lyophilized powder consists of the following:
Formulation per 2.0 ml dose:
3 MIU of IFN-b-1a
mannitol
HSA
Sodium acetate
pH 5.5
   The specific activity of Rebif interferon is 2.7×10⁸ units/mg, i.e., 270 MU of antiviral activity per milligram of IFN-b-1a protein. The patient reconstitutes the powder with a sodium chloride solution (0.9% NaCl) prior to injection subcutaneously three times a week. The formulation of liquid Rebif is as follows:
Formulation per 0.5 ml dose:
6 or 12 MIU IFN-b-1a
4 or 2 mg HSA
27.3 mg mannitol
0.4 mg sodium acetate
water for injection
   The liquid formulation is packaged in a pre-filled syringe and administered with or without use of an autoinjector device (Rebiject) 3 times (6 or 12 MIU, corresponding to 66 μg/week or 132 μg/week, respectively) per week subcutaneously.

In yet another embodiment, the IFN-β is BETASERON® (from Berlex), an IFN-β containing a cys-17 to ser mutation that is produced in E. coli. This non-glycosylated IFN-β is less potent than AVONEX® or REBIF® which are both produced in CHO cells. Doses are sold as 250 mcg (8 MIU) doses, both in lyophilized and liquid formulations, for injection subcutaneously every other day. BETAFERON® is another commercially available IFN-β, which can be administered subcutaneously, according to the manufacturer's instructions.

IFN-β or a variant thereof can also be administered together with a soluble IFN type I receptor or portion thereof, such as an IFN-binding chain of the receptor, as described, e.g., in U.S. Pat. No. 6,372,207. As described in the patent, administration of an IFN type I in the form of a complex with an IFN binding chain of the receptor improves the stability of the IFN and enhances the potency of the IFN. The complex may be a non-covalent complex or a covalent complex.

The IFN-β therapeutics may be tested in animal models of glomerulonephritis. Mammalian models of glomerulonephritis in, for example, mice, rats, guinea pigs, cats, dogs, sheep, goats, pigs, cows, horses, and non-human primates, may be created by causing an appropriate direct or indirect injury or insult to the renal tissues of the animal. Animal models of glomerulonephritis may, for example, be created by injecting antibodies to glomerular basement membrane, such as in the rat animal model nephrotoxic nephritis (NTN) described in the Examples. Other animal models may be created by injecting anti-Thy1 antibodies to the animals, as further described in the Examples. Yet other animal models are established by immunization with autologous glomerular basement membrane or by unilateral ureteric obstruction (UUO).

The IFN-β therapeutics may be evaluated for their therapeutic efficacy in causing a clinically significant improvement in a standard marker of renal function when administered to a mammalian subject (e.g., a human patient) having or at risk of developing glomerulonephritis or chronic renal failure. Such markers of renal function are well known in the medical literature and include, without being limited to, rates of increase in proteinuria, BUN levels, rates of increase in serum creatinine, static measurements of BUN, static measurements of serum creatinine, glomerular filtration rates (GFR), ratios of BUN/creatinine, serum concentrations of sodium (Na+), urine/plasma ratios for creatinine, urine/plasma ratios for urea, urine osmolality, daily urine output, and the like (see, for example, Brenner and Lazarus (1994), in Harrison's Principles of Internal Medicine, 13th edition.

The present invention is further illustrated by the following examples, which should not be construed as limiting in any way. The contents of all cited references (including literature references, issued patents, published patent applications as cited throughout this application) are hereby expressly incorporated by reference.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization(B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

### Example 1

### IFN-β Induces a Significant Decrease in Proteinuria in Renal Failure

This Example describes that IFN-β significantly reduces proteinuria in the rat animal model NTN (nephrotoxic nephritis), which is an inflammatory model that histologically closely resembles human crescentic glomerulonephritis, leading to chronic renal failure.

The disease is induced in the rats by i.v. injecting nephrotoxic (NTS) serum, that is produced by the immunisation of rabbits with a preparation of lyophilized rat glomerular basement membrane (GBM). The NTS rapidly binds to the GBM, which leads to a vigorous, intraglomerular inflammatory response with upregulation of pro-inflammatory cytokines and adhesion molecules. There is an influx of leukocytes into the glomerulus. Glomeruli then develop areas of necrosis with deposition of fibrin and the disruption of capillary loops. This leads to the development of crescents—accumulations of inflammatory cells and proliferating glomerular epithelial cells in Bowman's space. This inflammatory space is characterized by loss of large amounts of protein in the urine. The glomeruli develop progressive scarring with accumulation of collagen in the tuft and fibrous transformation of crescents. The rats then develop terminal renal failure. Thus, in this model, rats respond to anti-GBM antibodies with acute but transient renal disease and then 100% of the animals progress to CRF on a well-defined course. Different rat strains have varying susceptibility to this form of renal injury, the Wistra-Kyoto (WKY) rat being extremely sensitive. This animal model is further described, e.g., in Tam et al. (1999) Nephrol. Dial. Transplant. 14:1658 and Allen et al. (1999) J. Immunol. 162:5519.

IFN-β used in this study was rat IFN-β corresponding to amino acids 22-184 of GenBank Accession No. P70499. Rat IFN-β was expressed in Chinese Hamster Ovary (CHO) S-32 cells adapated to growth in suspension and secreted into the culture medium. The cells were grown in serum-containing medium in fermentor cultures. IFN-β was purified from conditioned culture medium using sequential chromatography on Pharmacia SP-Sepharose, Blue Sepharose, and Superose 12 resins, and Biorad Bio-Scale Ceramic Hydroxyapatite and Bio-Scale S resins. The IFN-β was then dialyzed extensively against 25 mM citrate/150 mM NaCl (pH 4.5) and filter-sterilized (0.2 μm). The IFN-β preparation was >99% pure as determined by densitometry of Coomassie-stained non-reducing SDS-PAGE gels. The specific activity was determined to be about 3×10⁸ units/mg as measured on rat RATEC cells.

In this Example, NTN was induced in 28 WKY rats obtained from Charles River Laboratories. Four rats were killed at day 14 for baseline histology and the others were randomised to receive either IFN-β 3×10⁵ units/day intraperitoneally (i.p.), IFN-β 6×10⁵ units/day i.p. or vehicle only. Injections were given 6 days per week and treatment was continued until day 30. Proteinuria was measured at day 7 and then weekly. Rats were bled at day 14, day 28 and at sacrifice. At the time of killing, kidney, lung, liver and spleen were fixed in formalin and kidney was snap frozen.

The functional parameters analyzed in this and/or subsequent Examples were assessed as follows:

Albuminuria/proteinuria: this reflects glomerular leakage and, to a lesser extent, failure of tubular metabolism of filtered protein. Interpretation of such data can be difficult since it is the product of two independent variables; increased GBM permeability leads to greater proteinuria but decreased glomerular filtration rate reduces glomerular proteinuria.

Urine was collected in metabolic cages 24 h prior to harvest. Urinary albumin concentration was determined by rocket immunoelectrophoresis. Urinary protein concentration was determined by sulphosalicylic acid precipitation.

Serum creatinine and creatinine clearance (CrCI): Peripheral blood was taken at harvest for determination of serum creatinine concentration using Olympus reagents and an Olympus AU600 analyzer (Olympus, Eastleigh, U.K.). Urinary creatinine concentration was also measured (Bayer RA-XT, Newbury, U.K.) to permit calculation of creatinine clearance.

Survival: The endpoint of these studies is either sudden death or killing to relieve distress. Animals are viewed daily by a blinded, independent observer and moribund animals killed as deemed necessary by the third party. In practice, about half of animals in survival studies reached the “killing” endpoint.

Hematoxylin and eosin stained sections were obtained for a crude assessment of glomerular scarring, tubular dropout interstitial inflammatory infiltrates and interstitial fibrosis using arbitrary scoring scales.

Glomerular fibrosis: the % renal cortical areas stained green by Masson-Trichrome histochemistry which offers a way of assessing collagen “load” within a kidney, was estimated by computer. Individual glomeruli can also be selected as the area of interest to calculate specific glomerular fibrosis. To quantify interstitial fibrosis within glomeruli, paraffin-embedded kidney sections were stained using a standard trichrome method (Martius Yellow, Brilliant Crystal Scarlet and Aniline Blue). To quantify glomerular fibrin deposition (e.g., fibrinoid necrosis), paraffin-embedded kidney sections were stained using Martius Yellow which stains fibrin a red/orange color. Sections were examined under X200 magnification using an Olympus BX40 microscope (Olympus Optical, London, U.K.) mounted with a Photonic digital camera (Photonic Science, East Sussex, U.K.). Images were captured and analyzed using Image-Pro Plus™ software (Media Cybernetics, Silver Spring, Md.).

Quantitation of the % renal cortical area stained brown after immunoperoxidase staining of kidney sections for the ED(A) domain of fibronectin appears to discriminate between CRF of differing functional severity. Similarly, type III collagen immunohistochemistry allows for calculation of the % renal cortical area stained.

Glomerular alpha-smooth muscle actin (SMA) expression was measured by immunofluorescence. This protein defines a population of “myofibroblastic” cells within glomeruli, which are thought to be key players in glomerular fibrosis. Quantitation of immunofluorescence staining for alpha-SMA correlates well with glomerular Masson-trichome fibrosis scores.

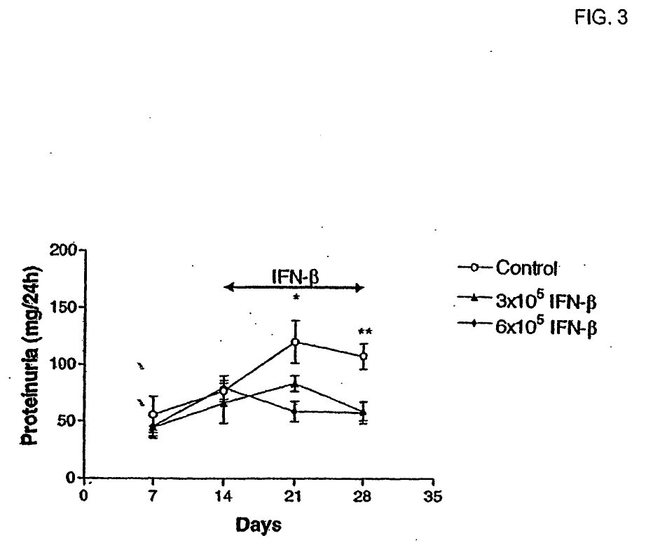

The results, which are shown in FIG. 3, indicate a marked reduction in proteinuria at day 21 and day 28 in the animals treated with both doses of IFN-β. There was no difference in serum creatine, creatine clearance, glomerular or tubulointerstitial scarring ranked on blinded H&E section (i.e., histological scarring); glomerular macrophages or CD8 numbers; or deposition of glomerular ED(A) fibronectin or type IV collagen.

### Example 2

### IFN-β Also Significantly Decreases Proteinuria During the Acute Phase of Renal Injury

This Example shows that, in addition to reducing proteinuria in later stages of renal failure, IFN-β also reduces proteinuria in the acute phase of renal injury.

For this example, NTN was induced in 32 rats, by injection of 0.1 ml NTS i.v., as described above. Eight rats were treated with rat IFN-β 6×10⁵ units/day i.p. 6 days per week from day 0 to day 14. Eight rats were treated with RSA i.p. 6 days per week from day 0 to day 14. Eight rats were treated with rat IFN-β 6×10⁵ units/day i.p. 6 days per week from day 0 to day 28. Eight rats were treated with RSA i.p. 6 days per week from day 0 to day 28. Urine was collected in metabolic cages from day 7, 14, 21 and 28 for the measurement of proteinuria and creatinine. All rats were bled on day 14 and at sacrifice for serum creatinine. Half of the rats of each group were killed at day 14 and half at day 28. One hour before the rats were killed at day 28, BrdU was injected for the assessment of cell proliferation. The following tissues were fixed in formalin for histology: kidney, lung, liver and spleen. Kidney sections were fixed in Camoy's fixative for BrdU staining. Kidney was also snap frozen Glomerular scarring, tubular atrophy and fibrosis were assessed semi-quantitatively in H&E stained sections.

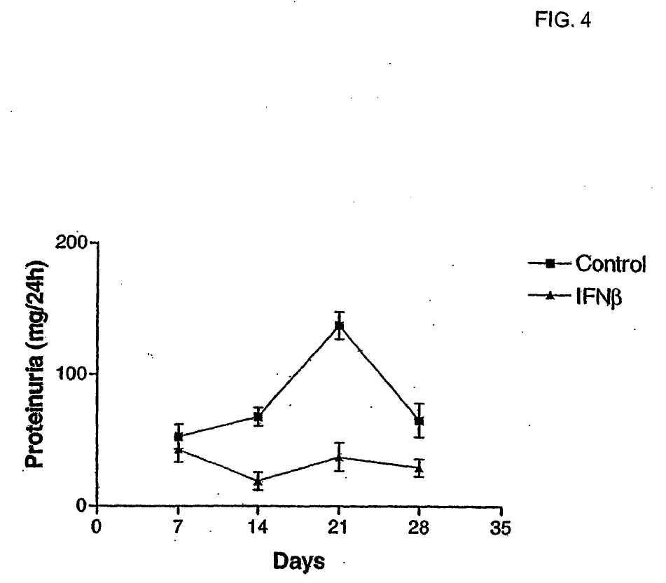

The results, which are shown in FIG. 4, indicate that IFN-β caused a marked reduction in proteinuria at days 14, 21 and 28. There were no differences in serum creatine and creatine clearance at days 14 and 28. Histologically, there was a significant reduction in glomerular macrophages (ED1+ cells) and CD8+ cells at day 14, but higher numbers at day 28. There was also a significant reduction in glomerular alpha-smooth muscle actin at day 28. Thus, INF-β treatment has an effect on proteinuria, reducing inflammation, but no apparent effect on scarring.

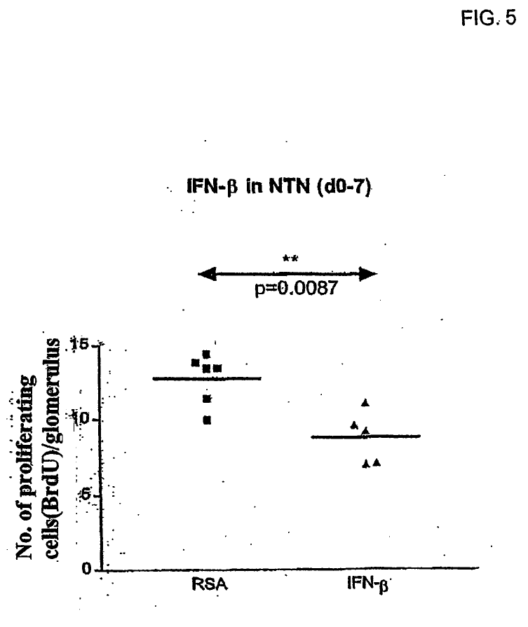

In another example, NTN was induced in 16 WKY rats, eight of which were treated with rat IFN-β 6×10⁵ units/day i.p. 6 days per week from day 0 to day 7 and the other eight of which were treated with vehicle (rat serum albumin-RSA) only, i.p. 6 days per week from day 0 to day 7. Rats were housed in metabolic cages on days 6 and 7. All rats were killed at day 7. One hour before killing the rats, they were injected with BrdU for assessement of cell proliferation. At the time of killing, kidney, lung, liver and spleen were fixed in formalin. Kidneys were fixed in Carnoy's fixative for BrdU staining and snap frozen. The results show that there does not appear to be a signficant difference in proteinuria, glomerular histology or glomerular macrophage or number of CD8 cells. However, fibrinoid score at day 7 was lower in the animals treated with IFN-β, relative to the control animals. In addition, the number of proliferating cells in the glomeruli was significantly lower in IFN-β treated animals relative to the control animals (see FIG. 5).

### Example 3

### IFN-β Induces a Significant Decrease in Proteinuria in the Renal Failure Animal Model Thy Glomerulonephritis

This Example shows that proteinuria is also significantly reduced by INF-β in mesangial proliferative glomerulonephritis.

For this example, the Thy1 glomerulonephritis animal model was used. This is an animal model of mesangial proliferative glomerulonephritis, which is characterized by increased proteinuria, mesangial cell proliferation and accumulation of mesangial matrix. This model depends on the fact that mesangial cells express the Thy1 antigen. Lewis rats are given a single i.v. injection of a monoclonal anti-Thy1 antibody. This leads to rapid and reproducible complement-mediated necrosis of glomerular mesangial cells (mesangiolysis). Proteinuria is apparent by 24 hours and persists for at least 10 days. Mesangiolysis is followed by a phase of repair in which mesangial cells proliferate and there is production of excess mesangial matrix. This is a reproducible model of proteinuria and mesangial cell proliferation.

Thy 1 glomerulonephritis was induced in 16 Lewis rats and 4 WKY rats by injection of 0.2 ml (2.5 mg/kg) anti-Thy1 antibody ER4. Eight Lewis rats received rat IFN-β 6×10⁵ units/day i.p., 6 days per week from day 0 to day 10. Eight Lewis rats received vehicle (rat serum albumin-RSA) alone, i.p., 6 days per week from day 0 to day 10. The four WKY rats received no treatment and the progression of the disease was observed from day 0 to day 10. Rats were housed in metabolic cages on days 6 and 7 and 9 and 10. Rats were killed on day 10. One hour before killing the rats, they were injected with BrdU for assessement of cell proliferation. At the time of killing, kidney, lung, liver and spleen were fixed in formalin. Kidneys were fixed in Carnoy's fixative for BrdU staining and snap frozen.

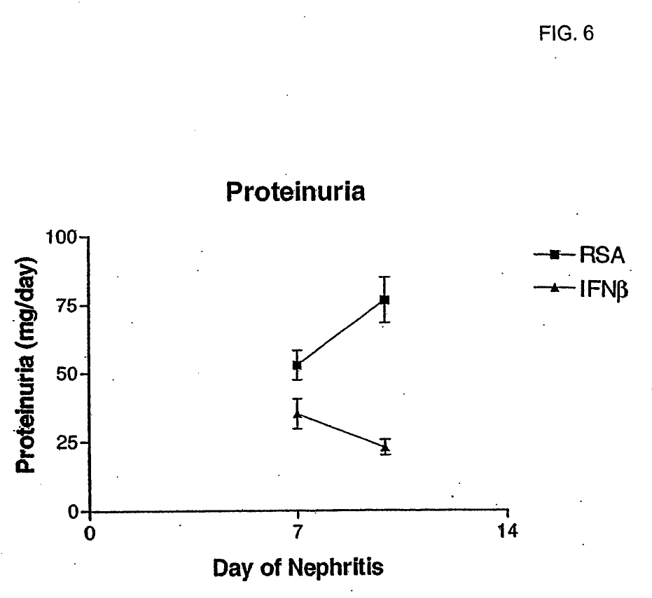

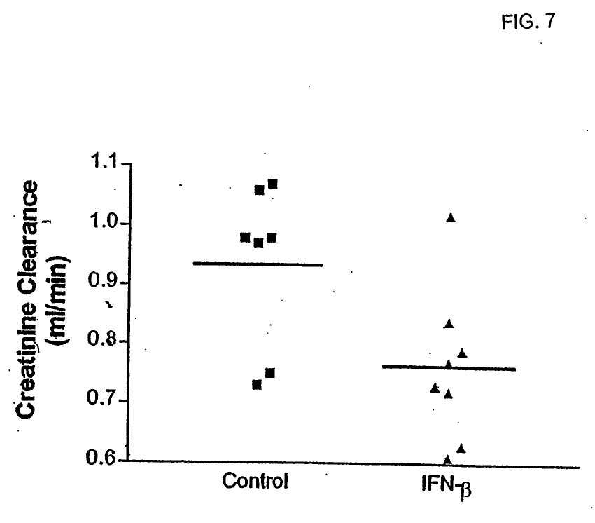

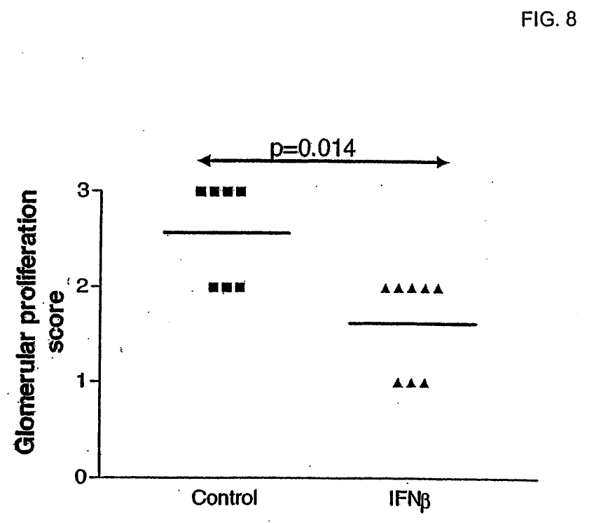

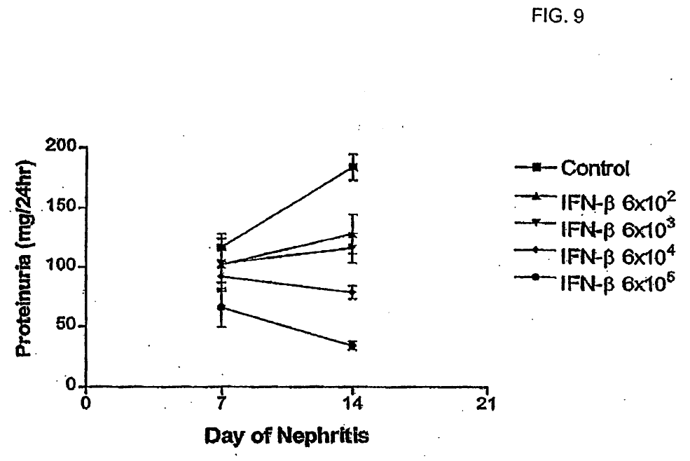

The results, which are shown in FIG. 6, indicate that proteinuria was significantly reduced at day 7 and at day 10. There does not appear to be any difference in serum creatinine, however, creatinine clearance showed a lower trend in the treated group (FIG. 7). There was no difference in acute glomerular injury as assessed by the presence of glomerular microaneurysms. However, glomerular hypercellularity was significantly reduced in rats treated with INF-β (FIG. 8).

### Example 4

### IFN-β Induces a Significant Decrease in Proteinuria in the Puromycin Aminonucleoside Nephropathy (PAN) Animal Model

PAN was induced in 4 male Wistar rats 200 g each. Two rats received 20 mg puromycin aminonucleoside (PA;) intraperiotenally (i.p.) on day 0 and two rats received 20 mg PA intra vascularly (i.v.) on day 0. Rats were housed in metabolic cages on days 3-4 and 7-8. All rats were killed on day 8. The results indicated that proteinuria had a mean value of 46 (mg/24 hours) and 287 at day 4 and day 8, respectively, in the rats injected i.p. and 122 and 194 at day 4 and day 8, respectively, in the rats injected i.v.

1-69. (canceled)

70. A method for treating glomerulonephritis in a mammal, comprising identifying a mammal having glomerulonephritis and administering to the mammal a therapeutically effective amount of an IFN-β therapeutic.

71. The method of claim 70, wherein glomerulonephritis is selected from the group consisting of focal glomeruloscerosis, collapsing glomerulopathies, minimal change disease, crescentic glomerulonephritis, nephritic syndrome, nephrotic syndrome, primary glomerulonephritis, secondary glomerulonephritis, proliferative glomerulonephritis, membraneous glomerulonephritis, membranoproliferative glomerulonephritis, immune-complex glomerulonephritis, anti-glomerular basement membrane (anti-GBM) glomerulonephritis, pauci-immune glomerulonephritis, diabetic glomerulopathy, chronic glomerulonephritis, and hereditary nephritis.

72. The method of claim 70, wherein the IFN-β therapeutic comprises mature IFN-β.

73. (canceled)

74. The method of claim 70, wherein the IFN-β is human IFN-β.

75. The method of claim 74, wherein the IFN-β is at least about 95% identical to full length mature human IFN-β having SEQ ID NO: 4.

76. The method of claim 75, wherein the IFN-β comprises SEQ ID NO: 4.

77. The method of claim 74, wherein the IFN-β is glycosylated.

78. (canceled)

79. The method of claim 74, wherein the IFN-β is IFN-β-1a.

80. The method of claim 74, wherein the IFN-β is IFN-β-1b.

81-84. (canceled)

85. The method of claim 70, wherein the IFN-β therapeutic comprises a pegylated IFN-β.

86-98. (canceled)

99. The method of claim 74, wherein the mammal is a human.

100-104. (canceled)

105. A method for treating chronic renal failure in a mammal, comprising identifying a mammal having chronic renal failure and administering to the mammal a therapeutically effective amount of an IFN-β therapeutic.

106. The method of claim 105, wherein the IFN-β therapeutic comprises mature IFN-β.

107. (canceled)

108. The method of claim 105, wherein the IFN-β is human IFN-β.

109. The method of claim 108, wherein the IFN-β is at least about 95% identical to full length mature human IFN-β having SEQ ID NO: 4.

110. The method of claim 109, wherein the IFN-β comprises SEQ ID NO: 4.

111. The method of claim 108, wherein the IFN-β is glycosylated.

112. (canceled)

113. The method of claim 108, wherein the IFN-β is IFN-β-1a.

114. The method of claim 108, wherein the IFN-β is IFN-β-1b.

115-118. (canceled)

119. The method of claim 105, wherein the IFN-β therapeutic comprises a pegylated IFN-β.

120-132. (canceled)

133. The method of claim 108, wherein the mammal is a human.

134-138. (canceled)


SEQUENCE LISTING (21 sequences)

SEQ ID NO: 1 - DNA, 840 bp, Homo sapiens
  CDS (76)..(636)
acattctaactgcaacctttcgaagcctttgctctggcacaacaggtagtaggcgacactgttcgtgttgtcaacatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtgcctctgggactggacaattgcttcaagcattcttcaaccagcagatgctgtttaagtgactgatggctaatgtactgcatatgaaaggacactagaagattttgaaatttttattaaattatgagttatttttatttatttaaattttattttggaaaataaattatttttggtgcaaaagtca

SEQ ID NO: 2 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 3 - DNA, 501 bp, Homo sapiens
  CDS (1)..(498)
atgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactga

SEQ ID NO: 4 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 5 - DNA, 15 bp, Artificial Sequence
  Description of Artificial Sequence Linker oligonucleotide
ggcggtggtggcagc

SEQ ID NO: 6 - protein, 5 aa, Artificial Sequence
  Description of Artificial Sequence Linker peptide
GGGGS

SEQ ID NO: 7 - DNA, 15 bp, Artificial Sequence
  Description of Artificial Sequence Enterokinase recognition site oligonucleotide
gacgatgatgacaag

SEQ ID NO: 8 - protein, 5 aa, Artificial Sequence
  Description of Artificial Sequence Enterokinase recognition site
DDDDK

SEQ ID NO: 9 - DNA, 24 bp, Artificial Sequence
  Description of Artificial Sequence Modified enterokinase recognition site oligonucleotide
agctccggagacgatgatgacaag

SEQ ID NO: 10 - protein, 8 aa, Artificial Sequence
  Description of Artificial Sequence Modified enterokinase recognition site
SSGDDDDK

SEQ ID NO: 11 - DNA, 1257 bp, Artificial Sequence
  CDS (1)..(1254)
  Description of Artificial Sequence Synthetic IFN-beta G162C-Ig direct fusion construct nucleotide sequence
atgcctgggaagatggtcgtgatccttggagcctcaaatatactttggataatgtttgcagcttctcaagccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacatgttacctccgaaacgtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 12 - protein, 418 aa, Artificial Sequence
  Description of Artificial Sequence Synthetic IFN-beta G162C-Ig direct fusion construct protein
MPGKMVVILGASNILWIMFAASQAMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTCYLRNVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1272 bp, Artificial Sequence
  CDS (1)..(1269)
  Description of Artificial Sequence Synthetic IFN-beta G162C-Ig fusion construct nucleotide sequence
atgcctgggaagatggtcgtgatccttggagcctcaaatatactttggataatgtttgcagcttctcaagccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacatgttacctccgaaacggcggtggtggcagcgtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 14 - protein, 423 aa, Artificial Sequence
  Description of Artificial Sequence Synthetic IFN-beta G162C-Ig fusion construct protein
MPGKMVVILGASNILWIMFAASQAMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTCYLRNGGGGSVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 18 bp, Artificial Sequence
  Description of Artificial Sequence 6-His oligonucleotide
catcatcatcatcatcat

SEQ ID NO: 16 - protein, 6 aa, Artificial Sequence
  Description of Artificial Sequence 6-His tag
HHHHHH

SEQ ID NO: 17 - DNA, 27 bp, Artificial Sequence
  Description of Artificial Sequence Modified His tag oligonucleotide
tccgggggccatcatcatcatcatcat

SEQ ID NO: 18 - protein, 9 aa, Artificial Sequence
  Description of Artificial Sequence Modified His tag
SGGHHHHHH

SEQ ID NO: 19 - DNA, 51 bp, Artificial Sequence
  Description of Artificial Sequence Modifed His tag oligonucleotide
tccgggggccatcatcatcatcatcatagctccggagacgatgatgacaag

SEQ ID NO: 20 - protein, 17 aa, Artificial Sequence
  Description of Artificial Sequence Modified His-tag
SGGHHHHHHSSGDDDDK

SEQ ID NO: 21 - protein, 8 aa, Artificial Sequence
  Description of Artificial Sequence Synthetic peptide epitope
DYKDDDDK